(19) United States
(12) Patent Application Publication
(10) Pub. No.: US 2021/0074431 A1
(43) Pub. Date: Mar. 11, 2021

(54) GENE EXPRESSION SUBTYPE ANALYSIS OF HEAD AND NECK SQUAMOUS CELL CARCINOMA FOR TREATMENT MANAGEMENT

(71) Applicants: GeneCentric Therapeutics, Inc., Research Triangle Park, NC (US); The University of North Carolina at Chapel Hill, Chapel Hill, NC (US)

(72) Inventors: Myla LAI-GOLDMAN, Durham, NC (US); Hawazin FARUKI, Durham, NC (US); Greg MAYHEW, Durham, NC (US); Charles PEROU, Carrboro, NC (US); David Neil HAYES, Chapel Hill, NC (US); Jose Zevallos, Research Triangle Park, NC (US)

(21) Appl. No.: 16/642,558

(22) PCT Filed: Aug. 30, 2018
(86) PCT No.: PCT/US2018/048862

§ 371 Date: Feb. 27, 2020

Related U.S. Application Data
(60) Provisional application No. 62/608,220, filed on Dec. 20, 2017.
(60) Provisional application No. 62/552,001, filed on Aug. 30, 2017.

(51) Int. Cl. G16H 50/30; C12Q 1/6886; G16H 50/70; G06F 17/15; G16B 40/10; G16B 40/20

(52) U.S. Cl. CPC G16H 50/30; C12Q 1/6886; G16H 50/70; C12Q 1/686; G16B 40/10; G16B 40/20; C12Q 2539/103; G06F 17/15

(57) ABSTRACT

Methods are provided for determining a subtype of head and neck squamous cell carcinoma (HNSCC) of an individual by detecting the expression level of at least one subtype classifier selected from a group of genes that are relevant for determining HNSCC subtypes. Also provided herein are methods for determining a suitable treatment and predicting the overall survival and the likelihood of metastasis for the HNSCC patients according to their subtypes.

### CROSS REFERENCE TO RELATED APPLICATIONS

This application claims the benefit of priority to U.S. Provisional Application No. 62/552,001 filed Aug. 30, 2017, and U.S. Provisional Application No. 62/608,220 filed Dec. 20, 2017, each of which is incorporated by reference herein in its entirety for all purposes.

### FIELD

The present disclosure relates to methods for determining a suitable treatment and predicting metastases and overall survival for a head and neck squamous cell carcinoma sample obtained from a patient having specific subtypes of head and neck cancer.

### BACKGROUND

Head and Neck Squamous Cell Carcinoma (HNSCC) is comprised of cancers arising from the oral cavity, oropharynx, nasopharynx, hypopharynx, and larynx and are responsible for approximately 3% of all malignancies. The most significant predisposing factors include heavy smoking and/or alcohol use, and more recently an increasing proportion of HNSCC tumors are caused by Human Papilloma Virus (HPV) Infection. In the United States, it is projected that in 2015, there were approximately 60,000 new cases and 12,000 deaths of HNSCCC (see Siegel R L, Miller K D, Jemal A. Cancer Statistics, 2015. CA Cancer J Clin. 2015; 65: 5-29). HNSCC has been traditionally managed with surgery, radiation therapy, and/or chemotherapy such that early stage tumors are often managed with a single treatment modality while advanced stage tumors require multimodality therapy. Risk stratification and treatment decisions vary by anatomic site, stage at presentation, histologic characteristics of the tumor, and patient factors.

Recent advances in cancer genomics have led to an increased understanding of mutational and gene expression profiles in HNSCC. HNSCC subtypes, as defined by underlying genomic features, have shown varied cell of origin, tumor drivers, proliferation, immune responses, and prognosis (Lawrence M S, Sougnez C, Lichtenstein L, Cibulskis K, Lander E, Gabriel S B, et al. Comprehensive genomic characterization of head and neck squamous cell carcinomas. Nature. 2015; 517: 576-582; Von Walter, Yin X, Wilkerson M D, Cabanski C R, Zhao N, Du Y, Ang M K, Hayward M C, Salazar A H, Hoadley K A, Fritchie K, Sailey C J, Weissler M C, Shockley W W, Zanation A M, Hackman T, Thorne L B, Funkhouser W D, Muldrew K L, Olshan A F, Randell S H, Wright F A, Shores C G, Hayes D N. (2013). Molecular Subtypes in Head and Neck Cancer Exhibit Distinct Patterns of Chromosomal Gain and Loss of Canonical Cancer Genes. PLoS One, 8(2):e56823; Keck M K, Zuo Z, Khattri a., Stricker T P, Brown C D, Imanguli M, et al. Integrative Analysis of Head and Neck Cancer Identifies Two Biologically Distinct HPV and Three Non-HPV Subtypes. Clin Cancer Res. 2014; 21: 870-881).

Currently, HNSCC tumors can be categorized into one of four subtypes (Atypical (AT), Mesenchymal (MS), Classical (CL), Basal (BA)). Each of these four subtypes can have distinct molecular signatures and varied mutational profiles (Chung C H, et al., Molecular classification of head and neck squamous cell carcinomas using patterns of gene expression. Cancer cell. May 2004; 5(5):489-500; Walter V, et al., Molecular subtypes in head and neck cancer exhibit distinct patterns of chromosomal gain and loss of canonical cancer genes. PloS one. 2013; 8(2):e56823). For example, the BA subtype can be characterized by over-expression of genes functioning in cell adhesion including COL17A1, and growth factor and receptor TGFA and EGFR. In another example, the CL subtype can be characterized by over-expression of genes related to oxidative stress response and xenobiotic metabolism, and can be most strongly associated with tobacco exposure. However, these distinct molecular characteristics of HNSCC have mostly not been incorporated into the patient treatment and risk management strategies, especially for HPV-negative HNSCC.

The present disclosure provides efficient methods for determining suitable treatments as well as the prognosis of nodal metastasis and overall survival for HNSCC patients according to their subtypes (e.g., AT, MS, CL and BA). The present disclosure also evaluates the likelihood of a HNSCC patient with a specific subtype responding to radiotherapy.

### SUMMARY OF THE INVENTION

In one aspect, provided herein is a method of determining a suitable treatment for a head and neck squamous cell carcinoma (HNSCC) patient, the method comprising: (a) detecting an expression level of at least one subtype classifier of from a publically available HNSCC dataset in a head and neck tissue sample obtained from the patient; and (b) selecting a treatment for the HNSCC patient according to the expression level of the at least one subtype classifier of the publically available HNSCC dataset; wherein the detection of the expression level of the subtype classifier specifically identifies a basal (BA), mesenchymal (MS), atypical (AT) or classical (CL) HNSCC subtype, and wherein the patient is HPV negative. In some cases, the expression level of the classifier biomarker is detected at the nucleic acid level. In some cases, the nucleic acid level is RNA or cDNA. In some cases, the detecting the expression level comprises performing quantitative real time reverse transcriptase polymerase chain reaction (qRT-PCR), gRT-PCR, RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing RNAseq. In some cases, the expression level is determined by RNAseq by Expected Maximization (RSEM). In some cases, the detecting the expression level comprises using at least one pair of oligonucleotide primers specific for at least one subtype classifier of the publically available HNSCC dataset. In some cases, the sample is a formalin-fixed, paraffin-embedded (FFPE) head and neck tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the at least one subtype classifier comprises a plurality of subtype classifiers. In some cases, the at least one subtype classifier comprises all the subtype classifiers of the publically available HNSCC dataset. In some cases, the HNSCC is oral cavity squamous cell carcinoma (OCSCC). In some cases, the HNSCC is laryngeal squamous cell carcinoma (LSCC). In some cases, the OCSCC is the MS subtype. In some cases, the OCSCC is the BA subtype. In some cases, the LSCC is the CL subtype. In some cases, the LSCC is the AT subtype. In some cases, the treatment comprises radiotherapy or surgery. In some cases, the method further comprises identifying resistance to radiotherapy. In some cases, the identifying comprises comparing the expression levels of the at least one subtype classifier of the publically available HNSCC dataset to expression levels of the at least one subtype classifier of the publically available HNSCC dataset in radiotherapy responder controls, radiotherapy non-responder controls or a combination thereof. In some cases, the identifying comprises measuring expression level of one or more genes in the KEAP1/NRF2 pathway. In some cases, the identifying comprises detecting a mutation in one or more genes in the KEAP1/NRF2 pathway. In some cases, the MS subtype is predictive of pathological nodal metastasis. In some cases, the subtype is predictive of overall survival of the patient. In some cases, the CL subtype in LSCC is predictive of a poor overall survival. In some cases, the publically available HNSCC dataset is the Cancer Genome Atlas (TCGA) HNSCC dataset. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or at least 840 subtype classifiers of the TCGA HNSCC dataset. In some cases, the publically available HNSCC dataset is a gene set comprising one or more of AKR1C1, NFE2L2, SOX2, KEAP1, RPA2, E2F2, FGFR3, PDGFRA, PDGFRB, TWIST1, EGFR, PIK3CA, TP63 and TGFA. In some cases, the publically available HNSCC dataset is the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or all 840 subtype classifiers of the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823. In some cases, the publically available HNSCC dataset is the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers or all 728 subtype classifiers of the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.

In another aspect, provided herein is a method of determining whether a HNSCC patient is likely to respond to radiotherapy, the method comprising: (a) detecting an expression level of at least one subtype classifier of a publically available HNSCC dataset in a head and neck tissue sample obtained from the patient, wherein the patient is HPV negative, and wherein the detection of the expression level of the subtype classifier specifically identifies a BA, MS, AT or CL HNSCC subtype; (b) determining expression of one or more genes associated with radiotherapy resistance; and (c) identifying the HNSCC subtype correlated with radiotherapy resistance. In some cases, the expression level of the subtype classifier is detected at the nucleic acid level. In some cases, the nucleic acid level is RNA or cDNA. In some cases, the detecting the expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques. In some cases, the expression level is detected by performing RNAseq. In some cases, the expression level is determined by RSEM. In some cases, the detecting the expression level comprises using at least one pair of oligonucleotide primers specific for the at least one subtype classifier of the publically available HNSCC dataset. In some cases, the sample is a FFPE head and neck tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the at least one subtype classifier comprises a plurality of subtype classifiers. In some cases, the at least one subtype classifier comprises all the subtype classifiers of the publically available HNSCC dataset. In some cases, the HNSCC is OCSCC. In some cases, the HNSCC is LSCC. In some cases, the OCSCC is the MS subtype. In some cases, the OCSCC is the BA subtype. In some cases, the LSCC is the CL subtype. In some cases, the LSCC is the AT subtype. In some cases, the HNSCC is the CL subtype. In some cases, the method further comprises comparing the expression levels of the at least one subtype classifier of the publically available HNSCC dataset between expression levels of the at least one subtype classifier of the publically available HNSCC dataset in radiotherapy responder controls and/or expression levels of the at least one subtype classifier of the publically available HNSCC dataset in radiotherapy non-responder controls. In some cases, the identifying comprises measuring expression level of one or more genes in the KEAP1/NRF2 pathway. In some cases, the identifying comprises detecting a mutation in one or more genes in the KEAP1/NRF2 pathway. In some cases, the publically available HNSCC dataset the Cancer Genome Atlas (TCGA) HNSCC dataset. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or at least 840 subtype classifiers of TCGA HNSCC dataset. In some cases, the publically available HNSCC dataset is a gene set comprising one or more of AKR1C1, NFE2L2, SOX2, KEAP1, RPA2, E2F2, FGFR3, PDGFRA, PDGFRB, TWIST1, EGFR, PIK3CA, TP63 and TGFA. In some cases, the publically available HNSCC dataset is the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or all 840 subtype classifiers of the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823. In some cases, the publically available HNSCC dataset is the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers or all 728 subtype classifiers of the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.

In yet another aspect, provided herein is a method of predicting occult nodal metastasis in a OCSCC patient, the method comprising: (a) detecting an expression level of at least one gene from a publically available HNSCC dataset in a head and neck tissue sample obtained from a patient, wherein the patient is HPV negative, wherein the detection of the expression level of the subtype classifier specifically identifies a BA, MS, AT or CL HNSCC subtype, and wherein identification of the MS subtype is indicative of occult nodal metastasis in the patient. In some cases, the expression level of the classifier biomarker is detected at the nucleic acid level. In some cases, the nucleic acid level is RNA or cDNA. In some cases, the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques. In some cases, the expression level is detected by performing RNAseq. In some cases, the expression level is determined by RSEM. In some cases, the detection of the expression level comprises using at least one pair of oligonucleotide primers specific for at least one subtype classifier of the publically available HNSCC dataset. In some cases, the sample is a FFPE head and neck tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the at least one subtype classifier comprises a plurality of subtype classifiers. In some cases, the at least one subtype classifier comprises all the subtype classifiers of the publically available HNSCC dataset. In some cases, the patient is suitable for neck dissection treatment. In some cases, the publically available HNSCC dataset the Cancer Genome Atlas (TCGA) HNSCC dataset. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or at least 840 subtype classifiers of TCGA HNSCC dataset. In some cases, the publically available HNSCC dataset is a gene set comprising one or more of AKR1C1, NFE2L2, SOX2, KEAP1, RPA2, E2F2, FGFR3, PDGFRA, PDGFRB, TWIST1, EGFR, PIK3CA, TP63 and TGFA. In some cases, the publically available HNSCC dataset is the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or all 840 subtype classifiers of the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823. In some cases, the publically available HNSCC dataset is the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers or all 728 subtype classifiers of the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.

In a still further aspect, provided herein is a method of predicting overall survival in a LSCC patient, the method comprising detecting an expression level of at least one gene from a publically available HNSCC dataset in a head and neck tissue sample obtained from a patient, wherein the patient is HPV negative, wherein the detection of the expression level of the subtype classifier specifically identifies a BA, MS, AT or CL LSCC subtype, and wherein identification of the LSCC subtype is predictive of the overall survival in the patient. In some cases, the expression level of the classifier biomarker is detected at the nucleic acid level. In some cases, the nucleic acid level is RNA or cDNA. In some cases, the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques. In some cases, the expression level is detected by performing RNAseq. In some cases, the expression level is determined by RSEM. In some cases, the detection of the expression level comprises using at least one pair of oligonucleotide primers specific for at least one subtype classifier of the publically available HNSCC dataset. In some cases, the sample is a FFPE head and neck tissue sample, fresh or a frozen tissue sample, an exosome, wash fluids, cell pellets, or a bodily fluid obtained from the patient. In some cases, the bodily fluid is blood or fractions thereof, urine, saliva, or sputum. In some cases, the at least one subtype classifier comprises a plurality of subtype classifiers. In some cases, the at least one subtype classifier comprises all the subtype classifiers of the publically available HNSCC dataset. In some cases, the method further comprises measuring the expression level of one or more genes in the KEAP1/NRF2 pathway. In some cases, the method further comprises detecting a mutation in one or more genes in the KEAP1/NRF2 pathway. In some cases, the LSCC subtype is the CL subtype, wherein the CL subtype is predictive of poor overall survival. In some cases, the patient is suitable for neck dissection treatment. In some cases, the publically available HNSCC dataset the Cancer Genome Atlas (TCGA) HNSCC dataset. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or at least 840 subtype classifiers of TCGA HNSCC dataset. In some cases, the publically available HNSCC dataset is a gene set comprising one or more of AKR1C1, NFE2L2, SOX2, KEAP1, RPA2, E2F2, FGFR3, PDGFRA, PDGFRB, TWIST1, EGFR, PIK3CA, TP63 and TGFA. In some cases, the publically available HNSCC dataset is the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, at least 728 subtype classifiers or all 840 subtype classifiers of the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823. In some cases, the publically available HNSCC dataset is the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are hereby incorporated by reference in their entirety for all purposes. In some cases, the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers or all 728 subtype classifiers of the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.

### BRIEF DESCRIPTION OF THE DRAWINGS

FIG. 1A illustrates the gene expression heat maps for each of the 4 subtypes (i.e., BA, MS, AT and CL) for the 728 gene set as described herein (i.e., Table 3, which is from Zevallos et al., Submitted as Thesis to Triological Society. 2017) for oral cavity squamous cell carcinoma (OCSCC) patients. FIG. 1B illustrates the gene expression heat maps for each of the 4 subtypes (i.e., BA, MS, AT and CL) for the 728 gene set as described herein (i.e., Table 3) for laryngeal squamous cell carcinoma (LSCC) patients.

FIG. 2A illustrates the gene expression heat maps for each of the 4 subtypes (i.e., BA, MS, AT and CL) for the set of 14 genes as described herein for OCSCC patients. FIG. 2B illustrates the gene expression heat maps including the set of 14 genes as described herein for LSCC patients. The set of 14 genes includes AKR1C1, NFE2L2, SOX2, KEAP1, RPA2, E2F2, FGFR3, PDGFRA, PDGFRB, TWIST1, EGFR, PIK3CA, TP63, and TGFA.

FIG. 3 illustrates Kaplan Meier 3-year survival curves for each of the 4 subtypes (i.e., BA, MS, AT and CL) for OCSCC. The BA subtype demonstrates the best 3-year survival rate (62.5%, 95% CI: 54.0%-72.4%) followed by AT subtype (51.5%, 95% CI: 35.2%-75.2%) and MS (47.3%, 95% CI: 37.5%-59.8%). The CL subtype has the worst 3-year survival (38.7%, 95% CI: 24.1%-62.1%).

FIG. 4 illustrates overall survival by gene expression in two early-stage OCSCC subtypes, BA and MS, respectively. The MS subtype is associated with worse overall 3-year survival rate compared to BA subtype (HR=3.86, 0.95-16.6, p=0.058).

FIG. 5 illustrates Kaplan Meier 3-year survival curves for each of the 4 subtypes (i.e., BA, MS, AT and CL) for LSCC. The AT subtype demonstrates the best 3-year survival rate (78.05%, 95% CI: 65.2%-93.2%). The CL subtype has the worst 3-year survival (43.7%, 95% CI: 30.0-63.7%). The BA and MS subtypes have similar survival rates (55.6%, 95% CI: 31.0%-99.7% and 58.3%, 95% CI: 41.1-82.5%, respectively).

FIG. 6 illustrates overall survival by gene expression in two LSCC subtypes, AT and CL, undergoing radiotherapy. The CL subtype is associated with worse overall 3-year survival rate compared to AT subtype (CL HR=3.30, 0.89-12.3, p=0.075).

FIG. 7A and FIG. 7B illustrate boxplots of expressions of Epithelial to Mesenchymal Transition (EMT) genes TWIST and Vimentin for each of the 4 subtypes (i.e., BA, MS, AT and CL). Both TWIST and Vimentin are significantly over-expressed in the MS subtype compared to AT and CL subtypes.

FIGS. 8A-8B illustrate the determination of the suitable treatments for the HNSCC patients by using the gene expression-based diagnostic assay. FIG. 8A shows that the T1-T2 node negative OCSCC patients are first categorized based on the invasiveness of the tumors (less than or more than 4 mm tumor depth) and within each invasiveness group, the patients are further categorized based on the risks (high versus low) of mesenchymal gene expressions. OCSCC patients who demonstrate high risks of mesenchymal gene expressions are assigned to neck dissection, whereas OCSCC patients who demonstrate low risks of mesenchymal gene expressions are assigned to routine observations and serial ultrasounds. FIG. 8B shows that surgically resectable HPV-negative HNSCC patients are first categorized into two groups based on the overall stages of their tumors (i-ii versus iii-iv). Patients within each group are then further categorized into either radiotherapy non-responders (Rad NR) or radiotherapy responders (Rad R). For HNSCC patients who have overall stages i-ii, the Rad NR are assigned to chemotherapy and radiation, whereas the Rad R are assigned to radiotherapy. For HNSCC patients who have overall stages iii-iv, the Rad R are assigned to chemotherapy and radiotherapy, whereas the Rad NR are assigned to surgery plus chemotherapy and radiotherapy.

### DETAILED DESCRIPTION

### Overview

The present disclosure provides methods for determining a suitable treatment for a HNSCC patient. The present disclosure provides methods for identifying or diagnosing HNSCC. That is, the methods can be useful for molecularly defining subtypes of HNSCC. The methods provide a classification of HNSCC subtypes that can be prognostic and predictive for therapeutic response. The present disclosure provides methods for selecting a suitable treatment for a HNSCC patient according to the classification of HNSCC. The present disclosure also provides methods for predicting metastasis in a HNSCC patient according to the classification of HNSCC. While a useful term for epidemiologic purposes, “Head and Neck Squamous Cell Carcinoma” can refer to cancers arising from the oral cavity, oropharynx, nasopharynx, hypopharynx, and larynx. Subtypes of these types of cancer as defined by underlying genomic features can have varied cell of origin, tumor drivers, proliferation, immune responses, and prognosis.

“Determining a HNSCC subtype” can include, for example, diagnosing or detecting the presence and type of HNSCC, monitoring the progression of the disease, and identifying or detecting cells, samples or expression of gene(s) that are indicative of subtypes.

In one embodiment, the suitable treatment is determined through evaluating the gene expression subtypes of HNSCC. In one embodiment, the gene expression subtype represents distinct molecular signatures. In one embodiment, HNSCC subtype is assessed through the evaluation of expression patterns, or profiles, of a plurality of subtype classifiers or biomarkers in one or more subject samples alone.

As described herein, the term subject, patient, or subject sample, refers to an individual regardless of health and/or disease status. A subject can be a subject, a study participant, a control subject, a screening subject, or any other class of individual from whom a sample is obtained and assessed in the context of the invention. Accordingly, a subject can be diagnosed with HNSCC (including subtypes, or grades thereof), can present with one or more symptoms of HNSCC, or a predisposing factor, such as a family (genetic) or medical history (medical) factor, for HNSCC, can be undergoing treatment or therapy for HNSCC, or the like. Alternatively, a subject can be healthy with respect to any of the aforementioned factors or criteria.

As used herein, the term “healthy” is relative to HNSCC status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more other cancers.

In one embodiment, the “expression level” “expression profile” or a “biomarker profile” “gene signature” or “molecular signature” associated with the subtype classifier described herein can be useful for determining HNSCC subtypes. In another embodiment, the tumor samples are HNSCC.

In one embodiment, HNSCC can be further identified as AT, BA, CL and MS based upon an expression profile determined using the methods provided herein. Expression profiles using the subtype classifiers disclosed herein can provide valuable molecular tools for specifically identifying HNSCC subtypes, and for determining a suitable treatment for a HNSCC patient. In some embodiments, the present method predicts therapeutic efficacy in treating HNSCC. Accordingly, the disclosure provides methods for classifying a subject for molecular HNSCC subtypes and methods for determining amenability of certain therapeutic treatments for HNSCC.

In some instances, a single subtype classifier or a plurality of subtype classifiers as provided herein is capable of identifying subtypes of HNSCC with a predictive success of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, about 93%, at least about 94%, at least about 95%, at least about 96%, at least about 97%, at least about 98%, at least about 99%, up to 100%.

In some instances, a single subtype classifier or a plurality of subtype classifiers as provided herein is capable of determining HNSCC subtypes with a sensitivity or specificity of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

In some embodiments, HNSCC described herein is oral cavity squamous cell carcinoma (OCSCC). In some embodiments, HNSCC described herein is laryngeal squamous cell carcinoma (LSCC). In some embodiments, HNSCC can be any type of head and neck malignancy.

As used herein, an “expression profile” or an “expression level” or a “subtype classifier profile” or a “gene signature” or a “molecular signature” comprises one or more values corresponding to a measurement of the relative abundance, level, presence, or absence of expression of subtype classifier or biomarker. An expression profile can be derived from a subject prior to or subsequent to a diagnosis of HNSCC, can be derived from a biological sample collected from a subject at one or more time points prior to or following treatment or therapy, can be derived from a biological sample collected from a subject at one or more time points during which there is no treatment or therapy (e.g., to monitor progression of disease or to assess development of disease in a subject diagnosed with or at risk for HNSCC), or can be collected from a healthy subject. The term subject can be used interchangeably with patient. The patient can be a human patient. The one or more subtype classifier provided herein is selected from a publically available HNSCC dataset in a head and neck tissue sample. The one or more subtype classifier provided herein is selected from the Cancer Genome Atlas (TCGA) head and neck cancer (HNSCC) dataset, the gene set provided in Walter V, Yin X, Wilkerson M D, et al. Molecular subtypes in head and neck cancer exhibit distinct patterns of chromosomal gain and loss of canonical cancer genes. PloS one. 2013; 8(2):e56823, Table 3 or any combination thereof. The one or more subtype classifier provided herein is selected from a gene set comprising one or more of AKR1C1, NFE2L2, SOX2, KEAP1, RPA2, E2F2, FGFR3, PDGFRA, PDGFRB, TWIST1, EGFR, PIK3CA, TP63 and TGFA.

As used herein, the term “determining an expression level” or “determining an expression profile” or “detecting an expression level” or “detecting an expression profile” as used in reference to a subtype classifier or biomarker means the application of a classifier specific reagent such as a probe, primer or antibody and/or a method to a sample, for example a sample of the subject or patient and/or a control sample, for ascertaining or measuring quantitatively, semi-quantitatively or qualitatively the amount of a classifier or classifiers, for example the amount of classifier polypeptide or mRNA (or cDNA derived therefrom). For example, a level of a classifier can be determined by a number of methods including for example immunoassays including for example immunohistochemistry, ELISA, Western blot, immunoprecipitation and the like, where a classifier detection agent such as an antibody for example, a labeled antibody, specifically binds the classifier and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR (qRT-PCR), gRT-PCR, serial analysis of gene expression (SAGE), Northern Blot, digital molecular barcoding technology, for example Nanostring Counter Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and this technology has been shown to be useful for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The expression profile or level of the subtype classifier can be used in combination with other diagnostic methods including histochemical, immunohistochemical, cytologic, immunocytologic, and visual diagnostic methods including histologic or morphometric evaluation of head and neck tissue.

In various embodiments of the present invention, the expression profile derived from a subject is compared to a reference expression profile. A “reference expression profile” or “control expression profile” can be a profile derived from the subject prior to treatment or therapy; can be a profile produced from the subject sample at a particular time point (usually prior to or following treatment or therapy, but can also include a particular time point prior to or following diagnosis of HNSCC); or can be derived from a healthy individual or a pooled reference from healthy individuals. A reference expression profile can be generic for HNSCC or can be specific to different subtypes of HNSCC. The HNSCC reference expression profile can be from the oral cavity, oropharynx, nasopharynx, hypopharynx, larynx or any combination thereof.

The reference expression profile can be compared to a test expression profile. A “test expression profile” can be derived from the same subject as the reference expression profile except at a subsequent time point (e.g., one or more days, weeks or months following collection of the reference expression profile) or can be derived from a different subject. In summary, any test expression profile of a subject can be compared to a previously collected profile from a subject that has an AT, MS, BL or CL HNSCC subtype. The previously collected profile can be HPV negative.

The subtype classifiers of the present disclosure can include nucleic acids (RNA, cDNA, and DNA) and proteins, and variants and fragments thereof. Such classifiers can include DNA comprising the entire or partial sequence of the nucleic acid sequence encoding the classifier, or the complement of such a sequence. The classifiers described herein can include RNA comprising the entire or partial sequence of any of the nucleic acid sequences of interest, or their non-natural cDNA products, obtained synthetically in vitro in a reverse transcription reaction. The biomarker nucleic acids can also include any expression product or portion thereof of the nucleic acid sequences of interest. A biomarker protein can be a protein encoded by or corresponding to a DNA biomarker of the invention. A classifier protein can comprise the entire or partial amino acid sequence of any of the classifier proteins or polypeptides. The classifier nucleic acid can be extracted from a cell or can be cell free or extracted from an extracellular vesicular entity such as an exosome.

A “subtype classifier” or “classifier biomarker” or “biomarker” or “classifier gene” can be any gene or protein whose level of expression in a tissue or cell is altered. For example, a “subtype classifier” or “classifier biomarker” or “biomarker” or “classifier gene” can be any gene or protein whose level of expression in a tissue or cell is altered in a specific HNSCC subtype. The detection of the subtype classifier of the present disclosure can permit the determination of the specific subtype. The “subtype classifier” or “classifier biomarker” or “biomarker” or “classifier gene” may be one that is up-regulated (e.g. expression is increased) or down-regulated (e.g. expression is decreased) relative to a reference or control as provided herein. The reference or control can be any reference or control as provided herein. In some embodiments, the expression levels of a “subtype classifier” or “classifier biomarker” or “biomarker” or “classifier gene” can be further compared between OCSCC, LSCC or any type of HNSCC.

In some embodiments, a publically available HNSCC dataset can be used for HNSCC subtype determination. In some embodiments, the publically available HNSCC dataset is the TCGA HNSCC dataset. In some embodiments, a total of 840 subtype classifiers obtained from TCGA HNSCC gene signature dataset can be used for HNSCC subtype determination. In one embodiment, a reduced set of 728 subtype classifiers (see Table 3) derived from the 840 subtype classifiers from TCGA HNSCC gene signature dataset can be used for HNSCC subtype determination. The TCGA HNSCC dataset includes at least 517 cases across all anatomic sites. In some embodiments, a set of 14 subtype classifier relevant to HNSCC can be used for HNSCC subtype determination (see Table 4). In another embodiment, any set of the subtype classifiers as described herein can be used for distinguishing the gene expression subtype of OCSCC and LSCC. In some embodiments, the publically available HNSCC dataset is the gene set found in Walter V, Yin X, Wilkerson M D, et al. Molecular subtypes in head and neck cancer exhibit distinct patterns of chromosomal gain and loss of canonical cancer genes. PloS one. 2013; 8(2):e56823, the contents of which are hereby incorporated by reference in their entirety for all purposes. In some embodiments, a total of 840 subtype classifiers obtained from the Walter et al. PloS one. 2013; 8(2):e56823 can be used for HNSCC subtype determination. In one embodiment, a reduced set of 728 subtype classifiers (Table 3) derived from the 840 subtype classifiers from the Walter et al. PloS one. 2013; 8(2):e56823 can be used for HNSCC subtype determination. In some cases, the publically available HNSCC dataset is the gene set found in Table 3, which is from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are hereby incorporated by reference in their entirety for all purposes.

In some embodiments, the gene expression subtype of HNSCC can determine or predict whether a patient would respond to a specific treatment. In some embodiments, the gene expression subtype of HNSCC can determine or predict whether a patient developed or is suspected of developing radiation resistance. In some embodiments, the gene expression subtype of HNSCC can determine or predict whether a patient would be suitable for a surgery. In some embodiments, the gene expression subtype of HNSCC can determine or predict the likelihood of a patient developing occult nodal metastases. In some embodiments, the gene expression subtype of HNSCC can determine or predict the overall survival rate of a HNSCC patient. In some embodiments, HNSCC is HPV-negative.

### HNSCC Subtyping and Gene Expression

In some embodiments, the methods provided herein allow for the determination of the four subtypes of HNSCC: (1) Basal (BA); (2) Mesenchymal (MS); (3) Atypical (AT); and (4) Classical (CL). In one embodiment, HNSCC is OCSCC. In one embodiment, HNSCC is LSCC. In one embodiment, HNSCC is any type of HNSCC. In some embodiments, the determination of the subtypes can serve as the guidance for treatment selections.

In general, the methods provided herein are used to classify HNSCC sample as a particular HNSCC subtype (e.g. subtype of HNSCC). In one embodiment, the method comprises measuring, detecting or determining an expression level of at least one of the subtype classifiers of any publically available HNSCC expression dataset. In one embodiment, the method comprises detecting or determining an expression level of at least one of the subtype classifiers of TCGA HNSCC gene signature dataset. The HNSCC sample for the detection or determination methods described herein can be a sample previously determined or diagnosed to be an HNSCC sample. In one embodiment, the HNSCC samples can be oral cavity clinical tumor samples. In one embodiment, the HNSCC samples can be tumors of larynx. In one embodiment, the HNSCC samples can be oropharynx cancer samples. In one embodiment, the HNSCC samples can be hypopharynx cancer samples. The previous diagnosis can be based on a histological analysis. The histological analysis can be performed by one or more pathologists.

In some embodiments, the methods provided herein are useful for determining the HNSCC subtype of a sample (e.g., head and neck tissue sample) from a patient by analyzing the expression of a set of subtype classifiers. The biomarkers or subtype classifiers useful in the methods provided herein can be selected from one or more HNSCC datasets from one or more databases. The databases can be public databases. In one embodiment, subtype classifiers useful in the methods provided herein for detecting or diagnosing HNSCC subtypes were selected from a HNSCC RNAseq dataset from TCGA. In some cases, the large set of subtype classifiers can be 840-gene classifier obtained from Walter et al. PloS one. 2013; 8(2):e56823 as described herein. In some cases, the large set of subtype classifiers can be the 728-gene classifier obtained from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017 as described herein, which is also referred to herein as Table 3. In some embodiments, the determination of a specific subtype can be determined by identifying the Nearest Centroid algorithm using a correlation-based similarity metric.

In some embodiments, the methods of the present disclosure require the detection of the expression level or abundance of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 728, at least 730, at least 740, at least 750, at least 760, at least 770, at least 780, at least 790, at least 800, at least 810, at least 820, at least 830, at least 840, inclusive of all ranges and subranges therebetween, of the genes present in the TGCA HNSCC dataset in a head and neck cancer cell sample obtained from a patient. In some embodiments, the methods of the present disclosure require the detection of the expression level or abundance of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 728, at least 730, at least 740, at least 750, at least 760, at least 770, at least 780, at least 790, at least 800, at least 810, at least 820, at least 830, at least 838, or at least 840, inclusive of all ranges and subranges therebetween, of the genes present in the Walter et al. PloS one. 2013; 8(2):e56823 gene set in a head and neck cancer cell sample obtained from a patient. In some embodiments, the methods of the present disclosure require the detection of the expression level or abundance of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, or at least 728, inclusive of all ranges and subranges therebetween, of the genes present in Table 3 (i.e., derived from Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017) in a head and neck cancer cell sample obtained from a patient. As provided herein, alteration of the expression level or abundance of the gene(s) form the TGCA HNSCC, Walter et al. PloS one. 2013; 8(2):e56823 or Table 3 (from Zevallos et al., Submitted as Thesis to Triological Society. 2017 dataset) can be used to identify a BA, MS, AT or CL HNSCC subtype. The same applies for other classifier gene expression datasets as provided herein.

In some embodiments, the genes used as subtype classifiers as used herein include a set of 14 genes (Table 4) relevant to HNSCC. In some embodiments, the set of 14 genes can include but is not limited to AKR1C1, NFE2L2, SOX2, KEAP1, RPA2, E2F2, FGFR3, PDGFRA, PDGFRB, TWIST1, EGFR, PIK3CA, TP63, and TGFA. In some embodiments, the methods of the present disclosure require the detection of the expression level or abundance of at least 1, at least 2, at least 3, at least 4, at least 5, at least 6, at least 7, at least 8, at least 9, at least 10, at least 11, at least 12, at least 13, at least 14 subtype classifiers of the set of genes in a head and neck cancer cell sample obtained from a patient. As provided herein, alteration of the expression level or abundance of the gene(s) can be used to identify a BA, MS, AT or CL HNSCC subtype. In some embodiments, a HNSCC subtype can be determined by analyzing any combination of the genes used as subtype classifiers from any of the publically available HNSCC datasets provided herein (e.g., TGCA HNSCC dataset, gene set from Walter et al. PloS one. 2013; 8(2):e56823, Table 3 and/or 14 gene HNSCC-related dataset) described herein that are suitable for subtype identification. By way of examples, a BA subtype can be determined by analyzing 60 subtype classifiers obtained from TCGA HNSCC dataset (or gene set from Walter et al. PloS one. 2013; 8(2):e56823 or Table 3) and 10 subtype classifiers obtained from the set of 14 genes as described herein. An AT subtype can be determined by analyzing 450 subtype classifiers obtained from TCGA HNSCC dataset (or gene set from Walter et al. PloS one. 2013; 8(2):e56823 or Table 3) and 10 subtype classifiers obtained from the set of 14 genes as described herein. In some embodiments, each HNSCC subtype can be determined by analyzing all 840 subtype classifiers from Walter et al. PloS one. 2013; 8(2):e56823 and the set of 14 subtype classifiers. In some embodiments, each HNSCC subtype can be determined by analyzing all 728 subtype classifiers from Table 3 and the set of 14 subtype classifiers (Table 4).

In some embodiments, the detecting includes all of the subtype classifiers of TCGA HNSCC gene signature dataset, gene set from Walter et al. PloS one. 2013; 8(2):e56823 or Table 3 at the nucleic acid level or protein level. In some embodiments, the detecting includes all of the subtype classifiers of the set of 14 genes (Table 4) relevant to HNSCC described herein at the nucleic acid level or protein level. In another embodiment, a single or a subset or a plurality of the subtype classifiers of TCGA HNSCC dataset gene signature, gene set from Walter et al. PloS one. 2013; 8(2):e56823 or Table 3 are detected. In another embodiment, a single or a subset or a plurality of the subtype classifiers of the set of 14 genes (Table 4) relevant to HNSCC described herein are detected. In yet another embodiment, a single or a subset or a plurality of the subtype classifiers of TCGA HNSCC dataset gene signature, gene set from Walter et al. PloS one. 2013; 8(2):e56823 or Table 3 are detected in combination with a single or a subset or a plurality of the subtype classifiers of the set of 14 genes (Table 4) relevant to HNSCC described herein.

It is recognized that additional genes or proteins can be used in the practice of the present disclosure. In general, genes useful in classifying the subtypes of HNSCC, include those that are independently capable of distinguishing between different classes or grades of HNSCC, or between different types of HNSCC. A gene can be considered to be capable of reliably distinguishing between subtypes if the area under the receiver operator characteristic (ROC) curve is approximately 1.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 728, at least 730, at least 740, at least 750, at least 760, at least 770, at least 780, at least 790, at least 800, at least 810, at least 820, at least 830, at least 840, inclusive of all ranges and subranges therebetween, of the genes present in the TCGA HNSCC dataset gene signature are “up-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, inclusive of all ranges and subranges therebetween, of the genes present in the TCGA HNSCC gene signature dataset are “down-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 728, at least 730, at least 740, at least 750, at least 760, at least 770, at least 780, at least 790, at least 800, at least 810, at least 820, at least 830, at least 840, inclusive of all ranges and subranges therebetween, of the genes present in the Walter et al. PloS one. 2013; 8(2):e56823 are “up-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, inclusive of all ranges and subranges therebetween, of the genes present in the Walter et al. PloS one. 2013; 8(2):e56823 are “down-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, or at least 728, inclusive of all ranges and subranges therebetween, of the genes present in Table 3 are “up-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 18, at least 19, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, or at least 728 inclusive of all ranges and subranges therebetween, of the genes present in Table 3 are “down-regulated” in a specific subtype of HNSCC (e.g., OCSCC or LSCC).

In some embodiments, at least 1, at least 2, at least 3, at least 4, at least 5, at least 6, at least 7, at least 8, at least 9, at least 10, at least 11, at least 12, at least 13, at least 14 subtype classifiers out of the set of 14 subtype classifiers are “up-regulated” in a specific subtype of HNSCC. In some embodiments, at least 1, at least 2, at least 3, at least 4, at least 5, at least 6, at least 7, at least 8, at least 9, at least 10, at least 11, at least 12, at least 13, at least 14 subtype classifiers out of the set of 14 subtype classifiers are “down-regulated” in a specific subtype of HNSCC (e.g., OCSCC or LSCC).

In some embodiments, a specific subtype of HNSCC (e.g., OCSCC or LSCC) can have a combination of up-regulated and down-regulated subtype classifiers. By way of examples, at least 50 subtype classifiers out of Table 3 can be up-regulated and at least 250 subtype classifiers out of Table 3 can be down-regulated for a specific subtype. In other examples, at least 300 subtype classifiers out of Table 3 can be up-regulated and at least 100 subtype classifiers out of Table 3 can be down-regulated for a specific subtype. In another example, at least 150 subtype classifiers out of Table 3 can be up-regulated, at least 450 subtype classifiers out of Table 3 can be down-regulated for a specific subtype, at least 10 subtype classifiers out of the set of 14 subtype classifiers can be up-regulated, and at least 4 subtype classifiers out of the set of 14 subtype classifiers can be down-regulated. In some embodiments, not all subtype classifiers described herein are required to be either up-regulated or down-regulated in a specific subtype of HNSCC. In some embodiments, the expression levels of certain subtype classifiers can be not altered. The same applies for any other subtype classifier gene expression datasets that can used for subtyping HNSCC (e.g., OCSCC or LSCC).

In some embodiments, the expression level of an “up-regulated” subtype classifier as provided herein is increased by about 0.5-fold, about 1-fold, about 1.5-fold, about 2-fold, about 2.5-fold, about 3-fold, about 3.5-fold, about 4-fold, about 4.5-fold, about 5-fold, inclusive of all ranges and subranges therebetween. In another embodiment, the expression level of a “down-regulated” subtype classifier as provided herein is decreased by about 0.5-fold, about 1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, inclusive of all ranges and subranges therebetween.

In some embodiments, the expression level of an “down-regulated” subtype classifier as provided herein is increased by about 0.5-fold, about 1-fold, about 1.5-fold, about 2-fold, about 2.5-fold, about 3-fold, about 3.5-fold, about 4-fold, about 4.5-fold, about 5-fold, inclusive of all ranges and subranges therebetween. In another embodiment, the expression level of a “down-regulated” subtype classifier as provided herein is decreased by about 0.5-fold, about 1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, inclusive of all ranges and subranges therebetween.

In one embodiment, the measuring or detecting step is at the nucleic acid level by performing RNA-seq, a reverse transcriptase polymerase chain reaction (RT-PCR) or a hybridization assay with oligonucleotides that are substantially complementary to portions of cDNA molecules of the at least one subtype classifier (such as the subtype classifiers of TCGA HNSCC gene signature dataset or Table 3) under conditions suitable for RNA-seq, RT-PCR or hybridization and obtaining expression levels of the at least one classifier biomarkers based on the detecting step. Each patient sample can then be assigned to one of the four subtypes of HNSCC according to the expression profiles of the subtype classifiers. In some embodiments, the subtypes can be determined by identifying the nearest centroid. In some embodiments, the identification can be achieved by using a correlation-based similarity metric. The subtype predictions in the test samples (e.g., HNSCC patient samples) can be determined by correlating each test sample with the subtype centroids as described herein and assigning the label of the centroid with the highest correlation.

The expression levels of the at least one of the subtype classifiers can then be compared to reference expression levels of the at least one of the subtype classifier biomarker from at least one sample training set. The at least one sample training set can comprise, (i) expression levels of the at least one subtype classifier from a sample that overexpresses the at least one subtype classifier, (ii) expression levels from a reference BA, MS, AT or CL sample, or (iii) expression levels from HNSCC free head and neck sample, and classifying the head and neck tissue sample as a BA, MS, AT or CL subtype. The head and neck cancer sample can then be classified as a BA, MS, AT or CL subtype of squamous cell carcinoma based on the results of the comparing step. In one embodiment, the comparing step can comprise applying a statistical algorithm which comprises determining a correlation between the expression data obtained from the head and neck tissue or cancer sample and the expression data from the at least one training set(s); and classifying the head and neck tissue or cancer sample as a BA, MS, AT or CL sample subtype based on the results of the statistical algorithm.

In one embodiment, the method comprises probing the levels of at least one of the subtype classifiers from a publically available database provided herein, such as, for example, the classifiers of TCGA HNSCC gene signature dataset or Table 3 at the nucleic acid level, in a head and neck cancer sample obtained from the patient. The probing step, in one embodiment, comprises mixing the sample with one or more oligonucleotides that are substantially complementary to portions of cDNA molecules of the at least one subtype classifier provided herein under conditions suitable for hybridization of the one or more oligonucleotides to their complements or substantial complements; detecting whether hybridization occurs between the one or more oligonucleotides to their complements or substantial complements; and obtaining hybridization values of the at least one subtype classifier based on the detecting step. The hybridization values of the at least one subtype classifier are then compared to reference hybridization value(s) from at least one sample training set.

The head and neck tissue sample can be any sample isolated from a human subject or patient. For example, in one embodiment, the analysis is performed on head and neck biopsies that are embedded in paraffin wax. In one embodiment, the sample can be a fresh frozen head and neck tissue sample. In another embodiment, the sample can be a bodily fluid obtained from the patient. The bodily fluid can be blood or fractions thereof (i.e., serum, plasma), urine, saliva, sputum or cerebrospinal fluid (CSF). The sample can contain cellular as well as extracellular sources of nucleic acid for use in the methods provided herein. The extracellular sources can be cell-free DNA and/or exosomes. In one embodiment, the sample can be a cell pellet or a wash. This aspect of the present disclosure provides a means to improve current diagnostics by accurately identifying the major histological types, even from small biopsies. The methods of the present disclosure, including the RT-PCR methods, are sensitive, precise and have multi-analyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. An advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

In one embodiment, the sample used herein is obtained from an individual, and comprises FFPE tissue. However, other tissue and sample types are amenable for use herein. In one embodiment, the other tissue and sample types can be fresh frozen tissue, wash fluids, or cell pellets, or the like. In one embodiment, the sample can be a bodily fluid obtained from the individual. The bodily fluid can be blood or fractions thereof (e.g., serum, plasma), urine, sputum, saliva or cerebrospinal fluid (CSF). A subtype classifier nucleic acid as provided herein can be extracted from a cell or can be cell free or extracted from an extracellular vesicular entity such as an exosome.

Methods are known in the art for the isolation of RNA from FFPE tissue. In one embodiment, total RNA can be isolated from FFPE tissues as described by Bibikova et al. (2004) American Journal of Pathology 165:1799-1807, herein incorporated by reference. Likewise, the High Pure RNA Paraffin Kit (Roche) can be used. Paraffin is removed by xylene extraction followed by ethanol wash. RNA can be isolated from sectioned tissue blocks using the MasterPure Purification kit (Epicenter, Madison, Wis.); a DNase I treatment step is included. RNA can be extracted from frozen samples using Trizol reagent according to the supplier's instructions (Invitrogen Life Technologies, Carlsbad, Calif.). Samples with measurable residual genomic DNA can be resubjected to DNaseI treatment and assayed for DNA contamination. All purification, DNase treatment, and other steps can be performed according to the manufacturer's protocol. After total RNA isolation, samples can be stored at −80° C. until use.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™. Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes).

In one embodiment, a sample comprises cells harvested from a head and neck tissue sample, for example, a squamous cell carcinoma sample. Cells can be harvested from a biological sample using standard techniques known in the art. For example, in one embodiment, cells are harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract nucleic acid, e.g, messenger RNA. All samples obtained from a subject, including those subjected to any sort of further processing, are considered to be obtained from the subject.

The sample, in one embodiment, is further processed before the detection of the subtype classifier levels of the combination of biomarkers set forth herein. For example, mRNA in a cell or tissue sample can be separated from other components of the sample. The sample can be concentrated and/or purified to isolate mRNA in its non-natural state, as the mRNA is not in its natural environment. For example, studies have indicated that the higher order structure of mRNA in vivo differs from the in vitro structure of the same sequence (see, e.g., Rouskin et al. (2014). Nature 505, pp. 701-705, incorporated herein in its entirety for all purposes).

mRNA from the sample in one embodiment, is hybridized to a synthetic DNA probe, which in some embodiments, includes a detection moiety (e.g., detectable label, capture sequence, barcode reporting sequence). Accordingly, in these embodiments, a non-natural mRNA-cDNA complex is ultimately made and used for detection of the biomarker. In another embodiment, mRNA from the sample is directly labeled with a detectable label, e.g., a fluorophore. In a further embodiment, the non-natural labeled-mRNA molecule is hybridized to a cDNA probe and the complex is detected.

In one embodiment, once the mRNA is obtained from a sample, it is converted to complementary DNA (cDNA) prior to the hybridization reaction or is used in a hybridization reaction together with one or more cDNA probes. cDNA does not exist in vivo and therefore is a non-natural molecule. Furthermore, cDNA-mRNA hybrids are synthetic and do not exist in vivo. Besides cDNA not existing in vivo, cDNA is necessarily different than mRNA, as it includes deoxyribonucleic acid and not ribonucleic acid. The cDNA is then amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material. The numbers of copies generated are far removed from the number of copies of mRNA that are present in vivo.

In one embodiment, cDNA is amplified with primers that introduce an additional DNA sequence (e.g., adapter, reporter, capture sequence or moiety, barcode) onto the fragments (e.g., with the use of adapter-specific primers), or mRNA or cDNA biomarker sequences are hybridized directly to a cDNA probe comprising the additional sequence (e.g., adapter, reporter, capture sequence or moiety, barcode). Amplification and/or hybridization of mRNA to a cDNA probe therefore serves to create non-natural double stranded molecules from the non-natural single stranded cDNA, or the mRNA, by introducing additional sequences and forming non-natural hybrids. Further, as known to those of ordinary skill in the art, amplification procedures have error rates associated with them. Therefore, amplification introduces further modifications into the cDNA molecules. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature, and (iv) the chemical addition of a detectable label to the cDNA molecules.

In some embodiments, the expression of a subtype classifier of interest is detected at the nucleic acid level via detection of non-natural cDNA molecules. The subtype classifiers described herein include RNA comprising the entire or partial sequence of any of the nucleic acid sequences of interest, or their non-natural cDNA product, obtained synthetically in vitro in a reverse transcription reaction. The term “fragment” is intended to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length subtype classifier polynucleotide disclosed herein. A fragment of a subtype classifier polynucleotide will generally encode at least 15, 25, 30, 50, 100, 150, 200, or 250 contiguous amino acids, or up to the total number of amino acids present in a full-length subtype classifier protein of the present disclosure.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their non-natural cDNA products). Normalization is performed to correct for or normalize away both differences in the amount of RNA or cDNA assayed and variability in the quality of the RNA or cDNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed subtype classifiers or a large subset thereof (global normalization approach).

Isolated mRNA can be used in hybridization or amplification assays that include, but are not limited to, Southern or Northern analyses, PCR analyses and probe arrays, NanoString Assays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least 7, 15, 30, 50, 100, 250, or 500 nucleotides in length and sufficient to specifically hybridize under stringent conditions to the non-natural cDNA or mRNA subtype classifier of the present disclosure.

As explained above, in one embodiment, once the mRNA is obtained from a sample, it is converted to complementary DNA (cDNA) in a hybridization reaction. Conversion of the mRNA to cDNA can be performed with oligonucleotides or primers comprising sequence that is complementary to a portion of a specific mRNA. Conversion of the mRNA to cDNA can be performed with oligonucleotides or primers comprising random sequence. Conversion of the mRNA to cDNA can be performed with oligonucleotides or primers comprising sequence that is complementary to the poly(A) tail of an mRNA. cDNA does not exist in vivo and therefore is a non-natural molecule. In a further embodiment, the cDNA is then amplified, for example, by the polymerase chain reaction (PCR) or other amplification method known to those of ordinary skill in the art. PCR can be performed with the forward and/or reverse primers comprising sequence complementary to at least a portion of a subtype classifier gene provided herein. The product of this amplification reaction, i.e., amplified cDNA is necessarily a non-natural product. As mentioned above, cDNA is a non-natural molecule. Second, in the case of PCR, the amplification process serves to create hundreds of millions of cDNA copies for every individual cDNA molecule of starting material. The number of copies generated is far removed from the number of copies of mRNA that are present in vivo.

In one embodiment, cDNA is amplified with primers that introduce an additional DNA sequence (adapter sequence) onto the fragments (with the use of adapter-specific primers). The adaptor sequence can be a tail, wherein the tail sequence is not complementary to the cDNA. For example, the forward and/or reverse primers comprising sequence complementary to at least a portion of a subtype classifier gene provided herein can comprise tail sequence. Amplification therefore serves to create non-natural double stranded molecules from the non-natural single stranded cDNA, by introducing barcode, adapter and/or reporter sequences onto the already non-natural cDNA. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (ii) adapter sequences are added to the ends of cDNA molecules to make DNA sequences that do not exist in vivo, (iii) the error rate associated with amplification further creates DNA sequences that do not exist in vivo, (iv) the disparate structure of the cDNA molecules as compared to what exists in nature, and (v) the chemical addition of a detectable label to the cDNA molecules.

In one embodiment, the synthesized cDNA (for example, amplified cDNA) is immobilized on a solid surface via hybridization with a probe, e.g., via a microarray. In another embodiment, cDNA products are detected via real-time polymerase chain reaction (PCR) via the introduction of fluorescent probes that hybridize with the cDNA products. For example, in one embodiment, biomarker detection is assessed by quantitative fluorogenic RT-PCR (e.g., with TaqMan® probes). For PCR analysis, well known methods are available in the art for the determination of primer sequences for use in the analysis.

Subtype classifiers provided herein in one embodiment, are detected via a hybridization reaction that employs a capture probe and/or a reporter probe. For example, the hybridization probe is a probe derivatized to a solid surface such as a bead, glass or silicon substrate. In another embodiment, the capture probe is present in solution and mixed with the patient's sample, followed by attachment of the hybridization product to a surface, e.g., via a biotin-avidin interaction (e.g., where biotin is a part of the capture probe and avidin is on the surface). The hybridization assay, in one embodiment, employs both a capture probe and a reporter probe. The reporter probe can hybridize to either the capture probe or the biomarker nucleic acid. Reporter probes e.g., are then counted and detected to determine the level of subtype classifier(s) in the sample. The capture and/or reporter probe, in one embodiment contain a detectable label, and/or a group that allows functionalization to a surface.

For example, the nCounter gene analysis system (see, e.g., Geiss et al. (2008) Nat. Biotechnol. 26, pp. 317-325, incorporated by reference in its entirety for all purposes, is amenable for use with the methods provided herein.

Hybridization assays described in U.S. Pat. Nos. 7,473,767 and 8,492,094, the disclosures of which are incorporated by reference in their entireties for all purposes, are amenable for use with the methods provided herein, i.e., to detect the subtype classifiers and classifier combinations described herein.

Subtype classifier levels may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934, each incorporated by reference in their entireties.

In one embodiment, microarrays are used to detect subtype classifier levels. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316, each incorporated by reference in their entireties. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992, each incorporated by reference in their entireties. Arrays can be packaged in such a manner as to allow for diagnostics or other manipulation of an all-inclusive device. See, for example, U.S. Pat. Nos. 5,856,174 and 5,922,591, each incorporated by reference in their entireties.

Serial analysis of gene expression (SAGE) in one embodiment is employed in the methods described herein. SAGE is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, Velculescu et al. Science 270:484-87, 1995; Cell 88:243-51, 1997, incorporated by reference in its entirety.

An additional method of subtype classifier level analysis at the nucleic acid level is the use of a sequencing method, for example, RNAseq, next generation sequencing, and massively parallel signature sequencing (MPSS), as described by Brenner et al. (Nat. Biotech. 18:630-34, 2000, incorporated by reference in its entirety). This is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

In some embodiments, the present disclosure can use RNA-seq by Expected Maximization (RSEM) to quantify gene expression levels from TCGA RNA-seq data. RSEM is a software tool for quantifying gene and isoform abundances from single-end or paired-end RNA-seq data. RSEM typically consists of two steps of analyses: (1) a set of reference transcript sequences (e.g., RSEM-prepare-reference) are generated and preprocessed for use by later RSEM steps; (2) a set of RNA-seq reads are aligned to the reference transcripts and the resulting alignments are used to estimate abundances and their credibility intervals (e.g., RSEM-calculate-expression). For the reference transcript sequences, a FASTA-formatted file of transcript sequences can be used. By way of examples, a file can be obtained from a reference genome database, a de novo transcriptome assembler, or an expressed sequence tag (EST) database. For the second step of analyses, the RSEM-calculate-expression script can handle both the alignment of reads against reference transcript sequences and the calculation of relative abundances. For example, RSEM can use the Bowtie alignment program to align reads, with parameters specifically chosen for RNA-seq quantification. The use of RSEM methods is described in Li et al., (BMC Bioinformatics, 2011, 12:323), which are incorporated by reference for those disclosures. In the present disclosure, the RSEM gene expression measurements for the HNSCC cases can be transformed using Log² (RSEM+1). The HNSCC cases can then be subsequently median centered by gene.

Another method of subtype classifier level analysis at the nucleic acid level is the use of an amplification method such as, for example, RT-PCR or quantitative RT-PCR (qRT-PCR). Methods for determining the level of biomarker mRNA in a sample may involve the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Numerous different PCR or qRT-PCR protocols are known in the art and can be directly applied or adapted for use using the presently described compositions for the detection and/or quantification of expression of discriminative genes in a sample. See, for example, Fan et al. (2004) Genome Res. 14:878-885, herein incorporated by reference. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR.

Quantitative RT-PCR (qRT-PCR) (also referred as real-time RT-PCR) is preferred under some circumstances because it provides not only a quantitative measurement, but also reduced time and contamination. As used herein, “quantitative PCR” (or “real time qRT-PCR”) refers to the direct monitoring of the progress of a PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. A DNA binding dye (e.g., SYBR green) or a labeled probe can be used to detect the extension product generated by PCR amplification. Any probe format utilizing a labeled probe comprising the sequences of the invention may be used.

Immunohistochemistry methods are also suitable for detecting the levels of the subtype classifiers of the present disclosure. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In some embodiments, the methods disclosed herein further identify OCSCC cases and LSCC cases among all HNSCC samples. As described herein in the present disclosure, the methods include analyzing the HNSCC cases by using publically available HNSCC dataset(s). In some embodiments, the methods include analyzing the HNSCC cases by using the TCGA HNSCC dataset. In some embodiments, the methods include analyzing the HNSCC cases by using the set of 14 genes (Table 4) as described herein. In some embodiments, the methods include analyzing the HNSCC cases by using the set of 728 genes from Table 3 as described herein. In some embodiments, the methods include analyzing the HNSCC cases by using the set of 840 genes from Von Walter et al. (PLoS One, 8(2):e56823) as described herein. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, inclusive of all ranges and subranges therebetween, of the OCSCC cases can have a BA subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, inclusive of all ranges and subranges therebetween, of the OCSCC cases can have a MS subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, inclusive of all ranges and subranges therebetween, of the OCSCC cases can have a CL subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, inclusive of all ranges and subranges therebetween, of the OCSCC cases can have a AT subtype.

In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, inclusive of all ranges and subranges therebetween, of the LSCC cases can have a AT subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, inclusive of all ranges and subranges therebetween, of the LSCC cases can have a CL subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19, at least 20%, at least 21%, at least 22%, at least 23%, at least 24%, at least 25%, inclusive of all ranges and subranges therebetween, of the LSCC cases can have a MS subtype. In some embodiments, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, inclusive of all ranges and subranges therebetween, of the LSCC cases can have a BA subtype.

In one embodiment, the OCSCC cases have about 42% BA subtype. In one embodiment, the OCSCC cases have about 34% MS subtype. In one embodiment, the OCSCC cases have about 14% CL subtype. In one embodiment, the OCSCC cases have about 12% AT subtype. In one embodiment, the OCSCC cases primarily have MS and BA subtypes. In one embodiment, the LSCC cases have about 35% AT subtype. In one embodiment, the LSCC cases have about 31% CL subtype. In one embodiment, the LSCC cases have about 22% MS subtype. In one embodiment, the LSCC cases have about 10% BA subtype. In one embodiment, the LSCC cases primarily have CL and AT subtypes. As described herein, Table 1 shows the demographic, tumor, and treatment characteristics of the OCSCC and LSCC cases by subtype.

TABLE 1
Descriptive statistics of clinical and demographic variables by subtype for each cancer site.

| | | Oral Cavity Cancer | | | | | | | | |
|---|---|---|---|---|---|---|---|---|---|---|
| | | Atypical | | Basal | | Classical | | Mesenchymal | | |
| | | n = 35 | | n = 128 | | n = 43 | | n = 103 | | |
| | | n (%) | | n (%) | | n (%) | | n (%) | | p-value |
| Pathologic | N0 | 13 | (41.9) | 55 | (50.9) | 21 | (50.0) | 28 | (33.3) | 0.019 |
| N Stage | N1 | 7 | (22.6) | 17 | (15.7) | 6 | (14.3) | 16 | (19.0) | |
| | N2 | 9 | (29.0) | 36 | (33.3) | 15 | (35.7) | 39 | (46.4) | |
| | N3 | 4 | | 20 | | 1 | | 19 | | |
| | Missing | | | | | | | | | |
| Pathologic | T1 | 5 | (15.2) | 12 | (10.1) | 1 | (2.3) | 11 | (11.5) | 0.178 |
| T Stage | T2 | 10 | (30.3) | 37 | (31.1) | 10 | (23.3) | 37 | (38.5) | |
| | T3 | 3 | (9.1) | 28 | (23.5) | 10 | (23.3) | 18 | (18.8) | |
| | T4 | 15 | (45.5) | 42 | (35.3) | 22 | (51.2) | 30 | (31.2) | |
| | Missing | 2 | | 9 | | 0 | | 7 | | |
| Race | American Indian | 0 | (0.0) | 1 | (0.8) | 0 | (0.0) | 0 | (0.0) | 0.028 |
| | Asian | 0 | (0.0) | 9 | (7.3) | 0 | (0.0) | 1 | (1.0) | |
| | Black | 3 | (9.1) | 6 | (4.8) | 7 | (16.3) | 5 | (5.0) | |
| | White | 30 | (90.9) | 108 | (87.1) | 36 | (83.7) | 94 | (94.0) | |
| | Missing | 2 | | 4 | | 0 | | 3 | | |
| Smoking | Current | 10 | (30.3) | 38 | (29.7) | 18 | (42.9) | 27 | (27.6) | 0.273 |
| | Former | 13 | (39.4) | 47 | (36.7) | 18 | (42.9) | 44 | (44.9) | |
| | Never | 10 | (30.3) | 43 | (33.6) | 6 | (14.3) | 27 | (27.6) | |
| | Missing | 2 | | 0 | | 1 | | 5 | | |
| Radiation | No | 6 | (46.2) | 19 | (43.2) | 6 | (31.6) | 11 | (35.5) | 0.753 |
| | Yes | 7 | (53.8) | 25 | (56.8) | 13 | (68.4) | 20 | (64.5) | |
| | Missing | 22 | | 84 | | 24 | | 72 | | |
| Clinical | N0 | 21 | (61.8) | 64 | (53.3) | 23 | (54.8) | 56 | (55.4) | 0.762 |
| N Stage | N1 | 4 | (11.8) | 25 | (20.8) | 7 | (16.7) | 17 | (16.8) | |
| | N2 | 8 | (23.5) | 31 | (25.8) | 12 | (28.6) | 27 | (26.7) | |
| | N3 | 1 | (2.9) | 0 | (0.0) | 0 | (0.0) | 1 | (1.0) | |
| | Missing | 1 | | 8 | | 1 | | 2 | | |
| Clinical | T1 | 3 | (8.8) | 5 | (4.1) | 2 | (4.8) | 6 | (5.9) | 0.509 |
| T Stage | T2 | 9 | (26.5) | 40 | (32.5) | 10 | (23.8) | 39 | (38.2) | |
| | T3 | 7 | (20.6) | 36 | (29.3) | 9 | (21.4) | 23 | (22.5) | |
| | T4 | 15 | (44.1) | 42 | (34.1) | 21 | (50.0) | 34 | (33.3) | |
| Gender | | 1 | | 5 | | 1 | | 1 | | |
| | Female | 7 | (20.0) | 46 | (35.9) | 10 | (23.3) | 38 | (36.9) | 0.125 |
| | Male | 28 | (80.0) | 82 | (64.1) | 33 | (76.7) | 65 | (63.1) | |

| | | Laryngeal Cancer | | | | | | | | |
|---|---|---|---|---|---|---|---|---|---|---|
| | | Atypical | | Basal | | Classical | | Mesenchymal | | |
| | | n = 48 | | n = 12 | | n = 38 | | n = 27 | | |
| | | n (%) | | n (%) | | n (%) | | n (%) | | p-value |
| Pathologic | N0 | 17 | (44.7) | 5 | (55.6) | 10 | (35.7) | 9 | (34.6) | 0.88 |
| N Stage | N1 | 5 | (13.2) | 0 | (0.0) | 3 | (10.7) | 4 | (15.4) | |
| | N2 | 16 | (42.1) | 4 | (44.4) | 14 | (50.0) | 12 | (46.2) | |
| | N3 | 0 | (0.0) | 0 | (0.0) | 1 | (3.6) | 1 | (3.8) | |
| | Missing | 10 | | 3 | | 10 | | 1 | | |
| Pathologic | T1 | 3 | (7.7) | 1 | (10.0) | 2 | (6.2) | 1 | (3.7) | 0.91 |
| T Stage | T2 | 3 | (7.7) | 1 | (10.0) | 5 | (15.6) | 3 | (11.1) | |
| | T3 | 12 | (30.8) | 2 | (20.0) | 10 | (31.2) | 5 | (18.5) | |
| | T4 | 21 | (53.8) | 6 | (60.0) | 15 | (46.9) | 18 | (66.7) | |
| | Missing | 9 | | 2 | | 6 | | 0 | | |
| Race | American Indian | 0 | (0.0) | 0 | (0.0) | 0 | (0.0) | 1 | (3.8) | 0.458 |
| | Asian | 0 | (0.0) | 0 | (0.0) | 1 | (2.8) | 0 | (0.0) | |
| | Black | 9 | (18.8) | 2 | (18.2) | 3 | (8.3) | 6 | (23.1) | |
| | White | 39 | (81.2) | 9 | (81.8) | 32 | (88.9) | 19 | (73.1) | |
| | Missing | 0 | | 1 | | 2 | | 1 | | |
| Smoking | Current | 29 | (61.7) | 5 | (41.7) | 11 | (29.7) | 14 | (53.8) | 0.084 |
| | Former | 17 | (36.2) | 6 | (50.0) | 23 | (62.2) | 9 | (34.6) | |
| | Never | 1 | (2.1) | 1 | (8.3) | 3 | (8.1) | 3 | (11.5) | |
| | Missing | 1 | | 0 | | 1 | | 1 | | |
| Radiation | No | 3 | (16.7) | 0 | (0.0) | 2 | (28.6) | 3 | (37.5) | 0.431 |
| | Yes | 15 | (83.3) | 4 | (100.0) | 5 | (71.4) | 5 | (62.5) | |
| | Missing | 30 | | 8 | | 31 | | 19 | | |
| Clinical | N0 | 19 | (42.2) | 5 | (55.6) | 22 | (57.9) | 12 | (46.2) | 0.665 |
| N Stage | N1 | 9 | (20.0) | 1 | (11.1) | 3 | (7.9) | 5 | (19.2) | |
| | N2 | 17 | (37.8) | 3 | (33.3) | 11 | (28.9) | 8 | (30.8) | |
| | N3 | 0 | (0.0) | 0 | (0.0) | 2 | (5.3) | 1 | (3.8) | |
| | Missing | 3 | | 3 | | 0 | | 1 | | |
| Clinical | T1 | 1 | (2.1) | 0 | (0.0) | 1 | (2.6) | 1 | (3.8) | 0.504 |
| T Stage | T2 | 7 | (14.9) | 1 | (10.0) | 10 | (26.3) | 2 | (7.7) | |
| | T3 | 20 | (42.6) | 3 | (30.0) | 10 | (26.3) | 7 | (26.9) | |
| | T4 | 19 | (40.4) | 6 | (60.0) | 17 | (44.7) | 16 | (61.5) | |
| Gender | | | | | | | | | | |
| | Female | 7 | (20.0) | 46 | (35.9) | 10 | (23.3) | 38 | (36.9) | 0.125 |
| | Male | 28 | (80.0) | 82 | (64.1) | 33 | (76.7) | 65 | (63.1) | |

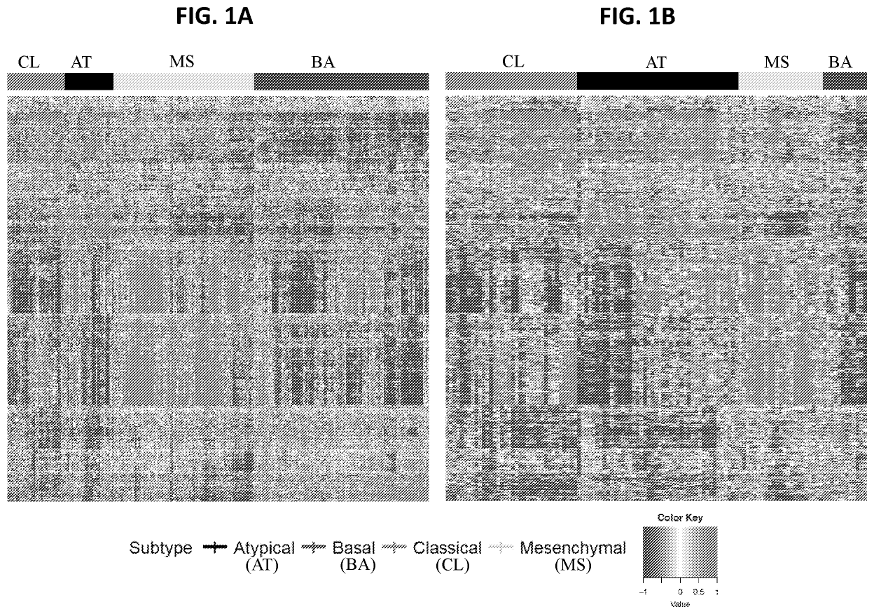

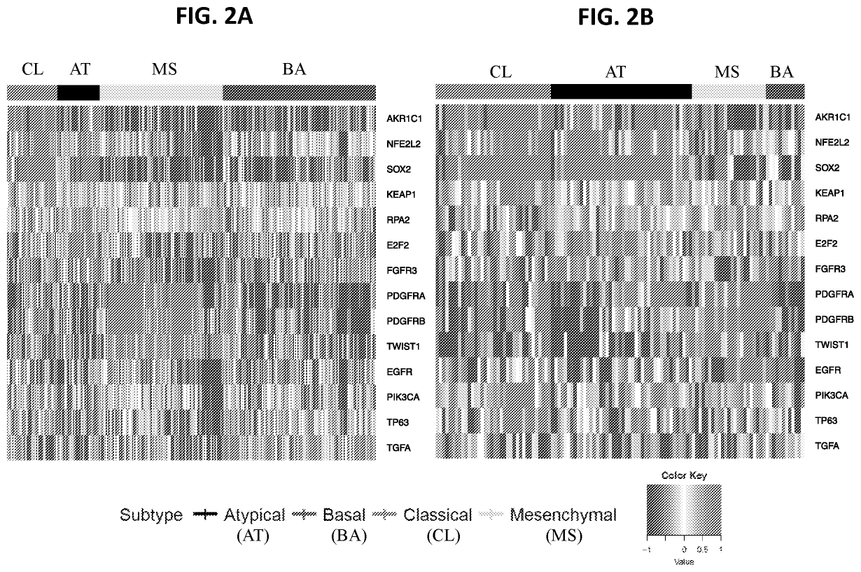

In some embodiments, MS subtype of OCSCC cases can be significantly more likely to be correlated with pathologically node positive compared to other subtypes among OCSCC cases. In some embodiments, at least about 10%, at least about 15%, at least about 20%, at least about 25%, at least about 30%, at least about 35%, at least about 40%, at least about 45%, at least about 50%, at least about 55%, at least about 60%, at least about 65%, inclusive of all ranges and subranges therebetween, MS subtypes of OCSCC are pathologically node positive. In one embodiment, at least about 65% MS subtypes of OCSCC are pathologically node positive. As described herein, OCSCC and LSCC gene expressions of the 728 subtype classifiers (Table 3) derived from the 840 subtype classifiers from TCGA HNSCC gene signature dataset are shown in FIG. 1A and FIG. 1B. OCSCC and LSCC gene expressions of the 14 subtype classifiers (Table 4) related to HNSCC are shown in FIG. 2A and FIG. 2B.

Epithelial to Mesenchymal transition is a complex multistep process by which epithelial malignancies undergo loss of cell adhesion, loss of polarity and cohesion, increased motility, and acquire a mesenchymal phenotype. Epithelial to mesenchymal transition are considered to be correlated to tumor invasiveness and lymph node metastasis in OCSCC. Without wishing to be bound by theory, OCSCC has strong association between decreased E-cadherin expression, increased p-Src, Vimentin expression and lymph node metastasis. For example, high expression of Vimentin can be associated with poor disease-specific survival in oral tongue squamous cell carcinoma. In another example, certain transcription factors can act as inducers of epithelial to mesenchymal transition in OCSCC. In some embodiments, the transcription factors can include Slug, Snail, and Twist1. In some embodiments, Twist1 overexpression can be characteristic of the OCSCC MS subtype. Without wishing to be bound by theory, Twist 1 upregulation can be associated with advanced stage tumors, lymph node and distant metastasis, and poor survival.

In some embodiments, Twist1 overexpression can be associated with at least about 0.1-fold, at least about 0.2-fold, at least about 0.3-fold, at least about 0.4-fold, at least about 0.5-fold, at least about 0.6-fold, at least about 0.7-fold, at least about 0.8-fold, at least about 0.9-fold, at least about 1.0-fold, at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, or at least about 3.0-fold increased risk of death of OCSCC patients compared to those without overexpression.

In some embodiments, LSCC CL subtype can be associated with overexpression of KEAP1 and NRF2. Without wishing to be bound by theory, the KEAP1/NRF2 pathway, an essential regulator of oxidative stress from reactive oxygen species and xenobiotics, can be a possible mechanism of chemoradiation resistance in multiple cancers including HNSCC. Loss of function mutations in the KEAP1 tumor suppressor gene and activating mutations in the KEAP1 binding domain of NFE2L2 can result in the constitutive activation of NRF2. As shown in FIG. 2B, LSCC CL subtype demonstrates overexpression of KEAP1 and NFE2L2. Constitutive activation of NRF2 in turn can have pro-tumorigenic effects, including inhibition of apoptosis, promotion of cell proliferation, and chemoresistance. Therefore, KEAP1/NRF2 can be associated with poor outcome of HNSCC.

In some embodiments, the BA subtype of HNSCC can correlate to overexpression of COL17A. In some embodiments, the BA subtype of HNSCC can correlate to overexpression of TGFA. In some embodiments, the BA subtype of HNSCC can correlate to overexpression of EGFR. In some embodiments, the BA subtype of HNSCC can correlate to overexpression of TP63. In some embodiments, the MS subtype can correlate to overexpression of genes involved in immune responses. In some embodiments, the MS subtype can be associated with VIM. In some embodiments, the MS subtype can be associated with DES. In some embodiments, the MS subtype can be associated with TWIST1. In some embodiments, the MS subtype can be associated with HGF. In some embodiments, the CL subtype can correlate to overexpression of genes related to oxidative stress response. In some embodiments, the CL subtype can correlate to overexpression of genes related to xenobiotic metabolism. In some embodiments, the CL subtype can correlate to overexpression of genes related to tobacco exposure. In some embodiments, the AT subtype can correlate to overexpression of CDKN2A. In some embodiments, the AT subtype can correlate to overexpression of LIG1. In some embodiments, the AT subtype can correlate to overexpression of RPA2. In some embodiments, the AT subtype can correlate to low expression of EGFR.

With regard to the methods of determining the gene expression, the levels of the subtype classifier provided herein, such as, for example, the classifiers of TCGA HNSCC gene signature dataset, Table 3 or Von Walter et al. (PLoS One, 8(2):e56823), can be normalized against the expression levels of all RNA transcripts or their non-natural cDNA expression products, or protein products in the sample, or of a reference set of RNA transcripts or a reference set of their non-natural cDNA expression products, or a reference set of their protein products in the sample. In some embodiments, the levels of the subtype classifiers provided herein, are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, HNSCC subtypes can be evaluated using levels of protein expression of one or more of the subtype classifiers provided herein. The level of protein expression can be measured using an immunological detection method. Immunological detection methods which can be used herein include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. Such assays are routine and well known in the art (see, e.g., Ausubel e t al, eds, 1994, Current Protocols in Molecular Biology, Vol. I, John Wiley & Sons, Inc., New York, which is incorporated by reference herein in its entirety).

In one embodiment, antibodies specific for subtype classifier proteins are utilized to detect the expression of a subtype classifier protein in a body sample. The method comprises obtaining a body sample from a patient or a subject, contacting the body sample with at least one antibody directed to a subtype classifier that is selectively expressed in head and neck cancer cells, and detecting antibody binding to determine if the subtype classifier is expressed in the patient sample. A preferred aspect of the present disclosure provides an immunocytochemistry technique for diagnosing HNSCC subtypes. One of skill in the art will recognize that the immunocytochemistry method described herein below may be performed manually or in an automated fashion.

As provided throughout, the methods set forth herein provide methods for determining the HNSCC subtype of a patient for determining a suitable treatment. Once the subtype classifier levels are determined, for example by measuring non-natural cDNA biomarker levels or non-natural mRNA-cDNA subtype classifier complexes, the subtype classifier levels are compared to reference values or a reference sample, for example with the use of statistical methods or direct comparison of detected levels, to make a determination of the HNSCC subtype. The reference sample can be an HNSCC-free sample, a HNSCC AT, a HNSCC BA, a HNSCC CL, a HNSCC MS sample or any combination thereof.

In one embodiment, a specified statistical confidence level may be determined in order to provide a confidence level regarding the HNSCC subtype. For example, it may be determined that a confidence level of greater than 90% may be a useful predictor of the HNSCC subtype. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

Determining the HNSCC subtype in some cases can be improved through the application of algorithms designed to normalize and or improve the reliability of the gene expression data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a gene expression profile or profiles, e.g., to determine the HNSCC subtype. The subtype classifier levels, determined by, e.g., microarray-based hybridization assays, sequencing assays, NanoString assays, etc., are in one embodiment subjected to the algorithm in order to classify the profile. Supervised learning generally involves “training” a classifier to recognize the distinctions among subtypes such as BA positive, MS positive, AT positive or CL positive, and then “testing” the accuracy of the classifier on an independent test set. Therefore, for new, unknown samples the classifier can be used to predict, for example, the class (e.g., BA vs. MS vs. AT vs. CL) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals, in one embodiment, are computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may in one embodiment be considered unreliable due to their aberrant hybridization propensity or secondary structure issues.

In some embodiments of the present disclosure, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N−1) degrees of freedom. (N−1)*Probe-set Variance/(Gene Probe-set Variance). Chi-Sq(N−1) where N is the number of input CEL files, (N−1) is the degrees of freedom for the Chi-Squared distribution, and the “probe-set variance for the gene” is the average of probe-set variances across the gene. In some embodiments of the present invention, probe-sets for a given mRNA or group of mRNAs may be excluded from further analysis if they contain less than a minimum number of probes that pass through the previously described filter steps for GC content, reliability, variance and the like. For example in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of subtype classifier level data analysis in one embodiment, further include the use of a feature selection algorithm as provided herein. In some embodiments of the present disclosure, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of subtype classifier level data analysis, in one embodiment, include the use of a pre-classifier algorithm. For example, an algorithm may use a specific molecular fingerprint to pre-classify the samples according to their composition and then apply a correction/normalization factor. This data/information may then be fed in to a final classification algorithm which would incorporate that information to aid in the final diagnosis.

Methods of subtype classifier level data analysis, in one embodiment, further include the use of a classifier algorithm as provided herein. In one embodiment of the present disclosure, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., of varying subtype classifier level profiles, and/or varying molecular subtypes of HNSCC (e.g., BA, MS, AT, CL) are selected based on statistical significance of the difference in biomarker levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of classifier level data are known in the art and have been described for example in Smyth, G. K. 2004 Stat. Appl. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the subtype classifier level profiling may provide a quantitative value or values indicative of one or more of the following: molecular subtype of HNSCC (e.g., BA, MS, AT, CL); the likelihood of the success of a particular therapeutic intervention, e.g., surgery or radiotherapy. In one embodiment, the data is presented directly to the physician in its most useful form to guide patient care, or is used to define patient populations in clinical trials or a patient population for a given medication. The results of the molecular profiling can be statistically evaluated using a number of methods known to the art including, but not limited to: the students T test, the two sided T test, Pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases, the results of the subtype classifier profiling assays, are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, a computer or algorithmic analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present disclosure, the results of the subtype classifier level profiling assays are presented as a report on a computer screen or as a paper record. In some embodiments, the report may include, but is not limited to, such information as one or more of the following: the levels of subtype classifiers as compared to the reference sample or reference value(s); the likelihood the subject will respond to a particular therapy, based on the subtype classifier level values and the HNSCC subtype and proposed therapies.

In one embodiment, the results of the gene expression profiling may be classified into one or more of the following: basal positive, mesenchymal positive, atypical positive or classical positive, basal negative, mesenchymal negative, atypical negative or classical negative; likely to respond to surgery (e.g., neck dissection), radiotherapy, immunotherapy or chemotherapy; unlikely to respond to surgery, radiotherapy, immunotherapy or chemotherapy; or combinations thereof.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combinations thereof.

It is intended that the methods described herein can be performed by software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

### Treatment Selection for HNSCC Patients

The present disclosure provides methods for determining a suitable treatment for a HNSCC patient. In some embodiments, the determination of a suitable treatment can involve obtaining a head and neck tissue sample for a HNSCC patient. In some embodiments, the HNSCC patients can have various stages of cancers. In some embodiments, a suitable treatment can be determined by detecting the expression level of at least one subtype classifier of a publically available head and neck cancer database. In some embodiments, a suitable treatment can be determined by detecting the expression level of any subtype classifiers that are relevant to HNSCC. In one embodiment, the subtype classifiers can be obtained from the TCGA HNSCC gene signature dataset as described herein. In one embodiment, the subtype classifiers can be obtained from a set of 14 subtype classifiers relevant to HNSCC as described herein. In one embodiment, the subtype classifiers can be obtained from the Von Walter et al. (PLoS One, 8(2):e56823) gene set as described herein. In one embodiment, the subtype classifiers can be obtained from Table 3 as described herein. In one embodiment, the 14 subtype classifiers can include but are not limited to AKR1C1, NFE2L2, SOX2, KEAP1, RPA2, E2F2, FGFR3, PDGFRA, PDGFRB, TWIST′, EGFR, PIK3CA, TP63, and TGFA. In some embodiments, the HNSCC is OCSCC. In some embodiments, the HNSCC is LSCC. In some embodiments, the HNSCC is HPV-negative.

In some embodiments, the determination of a suitable treatment can identify treatment responders. In some embodiments, the determination of a suitable treatment can identify treatment non-responders. In some embodiments, the suitable treatments can include but are not limited to radiotherapy (radiation therapy), surgery, immunotherapy, chemotherapy, target therapy, angiogenesis inhibitor therapy, or combinations thereof. In some embodiments, the suitable treatment can be any treatment or therapeutic methods that can be used for a HNSCC patient. In some embodiments, the radiotherapy can include but are not limited to proton therapy and external-beam radiation therapy. In some embodiments, the radiotherapy can include any types or forms of treatment that is suitable for HNSCC patients. In some embodiments, the surgery can include laser technology, excision, lymph node dissection or neck dissection, and reconstructive surgery. In some embodiments, the surgery approaches can include but are not limited to minimally invasive or endoscopic head and neck surgery (eHNS), Transoral Robotic Surgery (TORS), Transoral Laser Microsurgery (TLM), Endoscopic Thyroid and Neck Surgery, Robotic Thyroidectomy, Minimally Invasive Video-Assisted Thyroidectomy (MIVAT), and Endoscopic Skull Base Tumor Surgery. In some embodiments, the surgery can include any types of surgical treatment that is suitable for HNSCC patients. In one embodiment, the suitable treatment is radiotherapy. In one embodiment, the suitable treatment is surgery.

In some embodiments, the HNSCC subtype that has radiotherapy resistance can be a CL subtype. In some embodiments, the HNSCC subtype that has radiotherapy resistance can be a BA subtype. In some embodiments, the HNSCC subtype that has radiotherapy resistance can be a MS subtype. In some embodiments, the HNSCC subtype that has radiotherapy resistance can be an AT subtype. In some embodiments, the HNSCC subtype that has radiotherapy resistance can be any HNSCC subtypes. In one embodiment, the HNSCC subtype is a CL subtype. Radiotherapy resistance in any HNSCC subtype can be determined by measuring or detecting the expression levels of one or more genes known in the art and/or provided herein associated with or related to the presence of radiotherapy resistance. Association of a particular gene to radiotherapy resistance can be determined by examining expression of said gene in one or more patients known to be radiotherapy non-responders and comparing expression of said gene in one or more patients known to be radiotherapy responders.

In one embodiment, provided herein is a method for determining whether a HNSCC cancer patient is likely to respond to radiotherapy by determining the subtype of HNSCC of a sample obtained from the patient and, based on the HNSCC subtype, assessing whether the patient is likely to respond to radiotherapy. In another embodiment, provided herein is a method of selecting a patient suffering from HNSCC for radiotherapy by determining a HNSCC subtype of a sample from the patient and, based on the HNSCC subtype, selecting the patient for radiotherapy. The determination of the HNSCC subtype of the sample obtained from the patient can be performed using any method for subtyping HNSCC known in the art. The determination of the HNSCC subtype of the sample obtained from the patient can be performed using any method for subtyping HNSCC provided herein.

In one embodiment, the sample obtained from the patient has been previously diagnosed as having HNSCC, and the methods provided herein are used to determine the HNSCC subtype of the sample. The previous diagnosis can be based on a histological analysis. The histological analysis can be performed by one or more pathologists. In one embodiment, the HNSCC subtyping is performed via gene expression analysis of a set or panel of subtype classifier or subsets thereof in order to generate an expression profile. The gene expression analysis can be performed on a head and neck cancer sample (e.g., HNSCC sample) obtained from a patient in order to determine the presence, absence or level of expression of one or more subtype classifiers selected from a publically available head and neck cancer database described herein. The HNSCC subtype can be selected from the group consisting of BA, AT, MS or CL.

In one embodiment, the present disclosure further provides methods for determining a suitable treatment for a LSCC patient. In some embodiments, the LSCC patient is HPV-negative. In one embodiment, the present disclosure further provides methods for determining a suitable treatment for an OCSCC patient. In some embodiments, the OCSCC patient is HPV-negative.

In some embodiments, the present disclosure provides methods for determining the likelihood of a HNSCC patient responds to radiotherapy. In some embodiments, the present disclosure provides methods for classifying a HNSCC patient as a responder or a non-responder to radiotherapy. In some embodiments, the present disclosure provides comparing the expression levels of the at least one subtype classifier of the publically available HNSCC dataset between expression levels of the at least one subtype classifier of the publically available HNSCC dataset in radiotherapy responder controls and/or expression levels of the at least one subtype classifier of the publically available HNSCC dataset in radiotherapy non-responder controls. In some embodiments, the present disclosure provides methods for determining the likelihood of an OCSCC patient responds to radiotherapy. In one embodiment, the present disclosure provides methods for determining the likelihood of a LSCC patient responds to radiotherapy. In another embodiment, the present disclosure provides methods for determining the likelihood of a HPV-negative LSCC patient responds to radiotherapy. In another embodiment, the present disclosure provides methods for identifying a HPV-negative LSCC CL subtype as radiotherapy non-responder.

In one embodiment, the methods of the present disclosure find use in predicting response to different lines of therapies based on the subtype of HNSCC. In some embodiments, the methods for determining a suitable treatment can be achieved by subtyping HNSCC such as LSCC and OCSCC. In one embodiment, subtyping LSCC guides the selections of primary surgery and radiotherapy. In one embodiment, the LSCC is early to intermediate stage cancers. In some embodiments, certain subtypes of LSCC can be more amenable to surgical intervention. In some embodiments, certain subtypes of LSCC can benefit more from elective neck dissection. In some embodiments, certain subtypes of LSCC can be more amenable to radiotherapy. In some embodiments, certain subtypes of LSCC can have higher risks for radiotherapy failure. In one embodiment, LSCC CL subtype is associated with a higher risk of radiotherapy resistance compared to the non-CL subtype.

In some embodiments, the methods described herein provides radiotherapy response predictive assay. In some embodiments, the radiotherapy response predictive assay can guide the clinicians to administer other therapeutic approaches. In some embodiments, the subtyping can be achieved by detecting the expression level or abundance of at least one subtype classifier as described herein. The subtype classifier can be obtained from any publically available dataset. In some embodiments, the subtype classifier can be obtained from the TCGA HNSCC dataset or subset thereof as provided herein. In some embodiments, the subtype classifier can be obtained from the set of 14 genes (Table 4) relevant to HNSCC. In one embodiment, the subtype classifiers can be obtained from the Von Walter et al. (PLoS One, 8(2):e56823) gene set as described herein. In one embodiment, the subtype classifiers can be obtained from Table 3 as described herein. In another embodiment, the method of subtyping a HNSCC (e.g., OCSCC or LSCC) sample obtained from a subject entails detecting subtype classifiers from more than one publically available dataset. The more than one publically available dataset can be the TCGA HNSCC dataset (or Table 3 or the Von Walter et al. (PLoS One, 8(2):e56823) gene set) and the set of 14 genes (Table 4) relevant to HNSCC provided herein. In some embodiments, a set of subtype classifiers for performing the method provided herein include any genes that are implicated in radiotherapy resistance such as NFE2L2, KEAP1 and CUL3. In a further embodiment, the method of subtyping a HNSCC (e.g., OCSCC or LSCC) sample obtained from a subject entails detecting subtype classifiers from more than one publically available dataset as well as assessing the expression level or abundance of one or more genes implicated or previously shown to play a role in resistance to radiotherapy. Genes that are implicated in radiotherapy resistance can include NFE2L2, KEAP1 and CUL3. In some embodiments, clinical features of the HNSCC can also be included for determining the suitability for the radiotherapy.

As disclosed herein, the subtype classifiers panels, or subsets thereof, can be those disclosed in any publically available HNSCC gene expression dataset or datasets. In one embodiment, the HNSCC and the subtype panel or subset thereof can be, for example, the HNSCC gene expression dataset (n=134) disclosed in Keck et al. (Clin. Cancer Res. 2014; 21: 870-881.), the contents of which are herein incorporated by reference in its entirety. In one embodiment, the HNSCC and the subtype panel or subset thereof can be, for example, the HNSCC gene expression dataset (n=138) disclosed in Von Walter et al. (PLoS One, 8(2):e56823), the contents of which are herein incorporated by reference in its entirety. In one embodiment, the HNSCC and the subtype panel or subset thereof can be, for example, the HNSCC gene expression dataset (n=270) disclosed in Wichman et al. (Intl Jrnl Cancer 2015; 137: 2846-2857), the contents of which are herein incorporated by reference in its entirety. In one embodiment, the HNSCC and the subtype panel or subset thereof can be, for example, the HNSCC gene expression dataset disclosed in Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017, the contents of which are herein incorporated by reference in its entirety.

In one embodiment, the method comprises determining a subtype of a HNSCC sample and subsequently determining a level of gene signature of said subtype. In one embodiment, the gene signature can be determined by analyzing any of the subtype classifiers as described herein. In one embodiment, the gene signature can be determined by analyzing any of the subtype classifiers known in the art. In one embodiment, the subtype is determined by measuring the expression levels of one or more subtype classifiers using sequencing (e.g., RNASeq), amplification (e.g., qRT-PCR) or hybridization assays (e.g., microarray analysis) as described herein.

In one embodiment, the clinical features can include but are not limited to tumor size, nodal status and age. In some embodiments, the nodal status (stage) can include different status of primary tumor (T). In some embodiments, the nodal status (stage) can include different status of regional lymph nodes (N). In some embodiments, the nodal status (stage) can include different status of distant metastasis.

In some embodiments, radiotherapy resistance can be associated with certain gene signatures or the expression of particular genes. In some embodiments, radiotherapy resistance can be associated with the alterations of KEAP1 (Kelch-like ECH-associated protein 1)/NRF2 (nuclear factor E2-related factor 2) pathway. Further to this embodiment, radiotherapy resistance can be associated with the altered expression of NFE2L2, KEAP1, CUL3 or a combination thereof. The KEAP1/NRF2 pathway can be related to the protection of cells against oxidative and xenobiotic damage (e.g., cytoprotective mechanisms). Under unstressed conditions, NRF2 is constantly ubiquitinated by the CUL3-KEAP1 ubiquitin E3 ligase complex and rapidly degraded in proteasomes. Upon exposure to overproduction of electrophilic and oxidative stresses, for example, reactive cysteine residues of KEAP1 become modified, leading to a decline in the E3 ligase activity, stabilization of NRF2 and robust induction of a battery of cytoprotective genes. NRF2, a transcription factor, when the expression level is elevated, can promote cancer cell survival and proliferation. While transient activation of NRF2 can play protective roles in normal cells, constitutive activation of NRF2 can have pro-tumorigenic effects such as inhibition of apoptosis and promotion of cell proliferation. Accumulation of NRF2 in cancer cells can create environments conducive for cell growth and protects against oxidative stress, chemotherapeutic agents, and radiotherapy. In some embodiments, a method of determining a subtype of a particular HNSCC also entails assessing the function of the KEAP1/NRF2 pathway. Assessing the function can entail determining the expression level of one or genes of the pathway and/or determining the activity level of one or more genes in the pathway.

In some embodiments, upon determining a patient's HNSCC subtype, the HNSCC patients can be selected for any combinations of suitable therapies. For example, chemotherapy or drug therapy with a radiotherapy, a neck dissection with an immunotherapy or a chemotherapeutic agent with a radiotherapy. In some embodiments, immunotherapy, or immunotherapeutic agent can be a checkpoint inhibitor, monoclonal antibody, biological response modifier, therapeutic vaccine or cellular immunotherapy.

The methods of the present disclosure are also useful for evaluating clinical response to therapy, as well as for endpoints in clinical trials for efficacy of new therapies. The extent to which sequential diagnostic expression profiles move towards normal can be used as one measure of the efficacy of the candidate therapy.

### Prediction of Overall Survival Rate and Metastasis for HNSCC Patients

The present disclosure provides methods for predicting overall survival rate for a HNSCC patient. In some embodiments, the prediction of overall survival rate can involve obtaining a head and neck tissue sample for a HNSCC patient. In some embodiments, the HNSCC patients can have various stages of cancers. In some embodiments, the overall survival rate can be determined by detecting the expression level of at least one subtype classifier of a publically available head and neck cancer database or dataset. In some embodiments, an overall survival rate can be determined by detecting the expression level of any subtype classifiers that are relevant to HNSCC. In one embodiment, the subtype classifiers can be obtained from the TCGA HNSCC gene signature dataset for HNSCC as described herein. In one embodiment, the subtype classifiers can be obtained from a set of 14 subtype classifier relevant to HNSCC as described herein. In one embodiment, the subtype classifiers can be obtained from the Von Walter et al. (PLoS One, 8(2):e56823) gene set as described herein. In one embodiment, the subtype classifiers can be obtained from Table 3 as described herein. In one embodiment, the 14 subtype classifiers can include but are not limited to AKR1C1, NFE2L2, SOX2, KEAP1, RPA2, E2F2, FGFR3, PDGFRA, PDGFRB, TWIST1, EGFR, PIK3CA, TP63, and TGFA. In some embodiments, the HNSCC is OCSCC. In some embodiments, the HNSCC is LSCC. In some embodiments, the HNSCC is HPV-negative.

In some embodiments, the present disclosure further provide methods of predicting overall survival in a OCSCC patient. In some embodiments, the prediction includes detecting an expression level of at least one gene from a publically available HNSCC dataset in a head and neck tissue sample obtained from a patient. In some embodiments, the OCSCC is HPV negative. In some embodiments, the detection of the expression level of the subtype classifier specifically identifies a BA, MS, AT or CL OCSCC subtype. In some embodiments, the identification of the OCSCC subtype is indicative of the overall survival in the patient. A mesenchymal subtype of OCSCC as ascertained by measuring one or more subtype classifiers in a sample obtained from a OCSCC patient as provided herein can indicate a poor overall survival of a OCSCC patient as compared to patients with other subtypes of OCSCC.

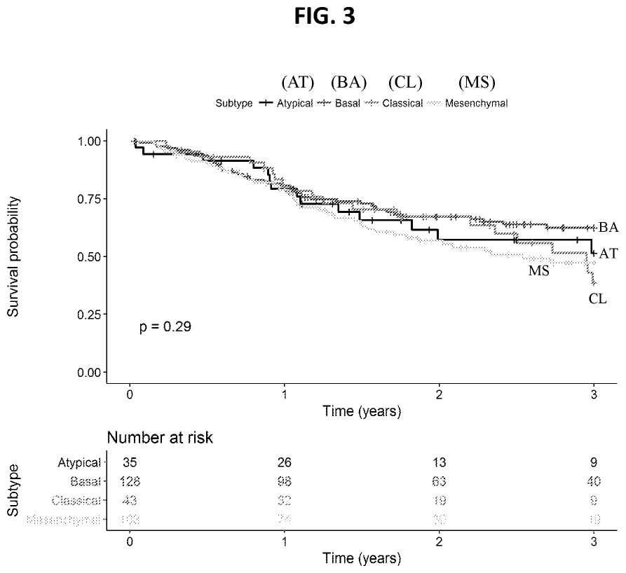

As shown in FIG. 3, OCSCC BA subtype can have the best 3-year survival compared to other subtypes. In some embodiments, the 3-year survival rate of the OCSCC BA subtype can be at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, or at least about 75%, inclusive of all ranges and subranges therebetween. In one embodiment, the 3-year survival rate of the OCSCC BA subtype is about 62.5%.

In some embodiments, the 3-year survival rate of OCSCC AT subtype can be at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, or at least about 60%, inclusive of all ranges and subranges therebetween. In one embodiment, the 3-year survival rate of the OCSCC AT subtype is about 51.5%.

In some embodiments, the 3-year survival rate of OCSCC MS subtype can be at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, or at least about 57%, inclusive of all ranges and subranges therebetween. In one embodiment, the 3-year survival rate of the OCSCC MS subtype is about 47.3%.

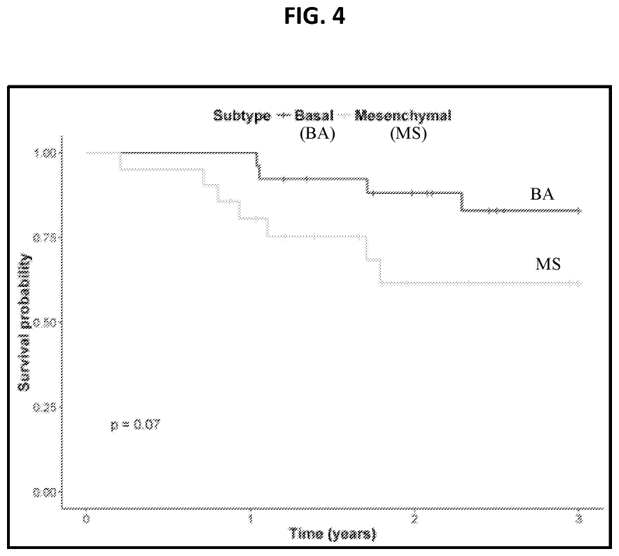

In some embodiments, the 3-year survival rate of OCSCC CL subtype can be at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, inclusive of all ranges and subranges therebetween. In one embodiment, the 3-year survival rate of the OCSCC CL subtype is about 43.7%. In another embodiment, as shown in FIG. 4, OCSCC MS subtype can be associated with worse overall survival compared to OCSCC BA subtype.

In some embodiments, the present disclosure further provide methods of predicting overall survival in a LSCC patient. In some embodiments, the prediction includes detecting an expression level of at least one gene from a publically available HNSCC dataset in a head and neck tissue sample obtained from a patient. In some embodiments, the LSCC is HPV negative. In some embodiments, the detection of the expression level of the subtype classifier specifically identifies a BA, MS, AT or CL LSCC subtype. In some embodiments, the identification of the LSCC subtype is indicative of the overall survival in the patient. A classical subtype of LSCC as ascertained by measuring one or more subtype classifiers in a sample obtained from a LSCC patient as provided herein can indicate a poor overall survival of a LSCC patient as compared to patients with other subtypes of LSCC.

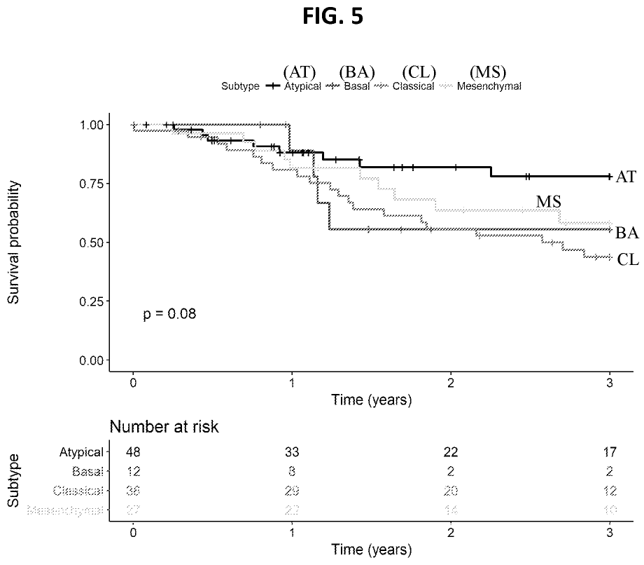

As shown in FIG. 5, LSCC AT subtype can have the best 3-year survival compared to other subtypes. In some embodiments, the 3-year survival rate of the LSCC AT subtype can be at least about 65%, at least about 66%, at least about 67%, at least about 68%, at least about 69%, at least about 70%, at least about 71%, at least about 72%, at least about 73%, at least about 74%, or at least about 75%, at least about 76%, at least about 77%, at least about 78%, at least about 79%, at least about 80%, at least about 81%, at least about 82%, at least about 83%, at least about 84%, at least about 85%, inclusive of all ranges and subranges therebetween. In one embodiment, the 3-year survival rate of the LSCC AT subtype is about 78.05%.

In some embodiments, the 3-year survival rate of LSCC BA subtype can be at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, or at least 65%, inclusive of all ranges and subranges therebetween. In one embodiment, the 3-year survival rate of the LSCC BA subtype is about 55.6%.

In some embodiments, the 3-year survival rate of LSCC MS subtype can be at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least 65%, at least 66%, at least 67%, or at least 68%, inclusive of all ranges and subranges therebetween. In one embodiment, the 3-year survival rate of the LSCC MS subtype is about 58.3%.

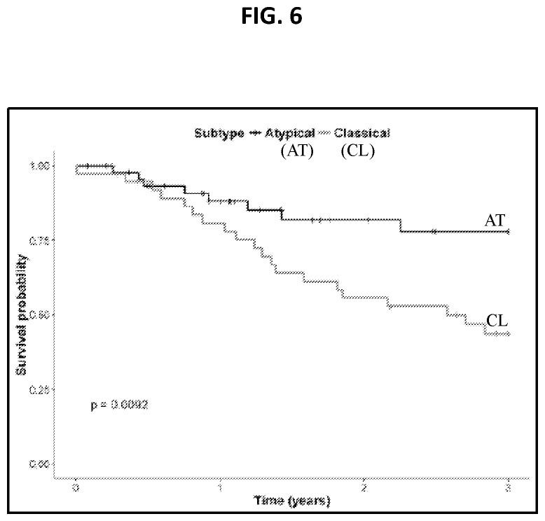

In some embodiments, the 3-year survival rate of LSCC CL subtype can be at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, or at least about 55%, inclusive of all ranges and subranges therebetween. In one embodiment, the 3-year survival rate of the LSCC CL subtype is about 47.3%. In another embodiment, as shown in FIG. 6, LSCC CL subtype can be associated with worse overall survival compared to LSCC AT subtype.

As described herein, Table 2 shows the multivariate regression analysis for factors associated with risk or death in OCSCC and LSCC cases. In some embodiments, the risks of death among all OCSCC subtypes do not significantly differ. In some embodiments, the risks of death among all OCSCC subtypes can significantly differ. As used herein, the term “significantly differ” can mean “significantly higher” or “significantly higher” or “positively associated” or “negatively associated.” For example, the risks of death of an OCSCC BA subtype can be significantly higher when compared to an OCSCC AT subtype. In some embodiments, the risks of death among all LSCC subtypes can significantly differ. In one embodiment, the LSCC CL subtype has an increased risk of death when compared to the LSCC AT subtype. In one embodiment, the LSCC MS subtype is associated with an increased risk of death when compared to the LSCC AT subtype. In some embodiments, the risks of death among all LSCC subtypes do not significantly differ.

In some embodiments, gender can be associated with the risks of death of HNSCC patients. In some embodiments, gender can be positively associated with the risks of death in OCSCC patients. In some embodiments, gender can be negatively associated with the risks of death in OCSCC patients. In some embodiments, gender can be not associated with the risks of death in OCSCC patients. In some embodiments, gender can be positively associated with the risks of death in LSCC patients. In some embodiments, gender can be negatively associated with the risks of death in LSCC patients. In some embodiments, gender can be not associated with the risks of death in LSCC patients. In one embodiment, female gender is associated with significantly worse survival compared to male gender in LSCC patients.

TABLE 2
Adjusted hazard ratios for oral cavity and laryngeal
cancers (CI: Confidence interval; HR: Hazards ratio).

| | Oral Cavity | | | Laryngeal | | |
|---|---|---|---|---|---|---|
| | HR (95% CI) | | p-value | HR (95% CI) | | p-value |
| Subtype | | | | | | |
| Atypical | 1.00 | | | 1.00 | | |
| Basal | 0.70 | (0.37, 1.32) | 0.265 | 0.93 | (0.18, 4.83) | 0.935 |
| Classical | 0.91 | (0.44, 1.86) | 0.793 | 4.32 | (1.77, 10.54) | 0.001 |
| Mesenchymal | 1.05 | (0.56, 1.96) | 0.888 | 2.51 | (0.91, 6.91) | 0.076 |
| Stage | | | | | | |
| IV | 1.00 | | | 1.00 | | |
| I-II | 0.83 | (0.53, 1.3) | 0.415 | 0.89 | (0.25, 3.17) | 0.864 |
| III | 1.03 | (0.64, 1.68) | 0.893 | 0.98 | (0.41, 2.38) | 0.973 |
| Gender | | | | | | |
| Male | 1.00 | | | 1.00 | | |
| Female | 1.13 | (0.75, 1.69) | 0.558 | 4.2 | (1.99, 8.90) | <0.001 |
| Race | | | | | | |
| White | 1.00 | | | 1.00 | | |
| Non-White | 1.36 | (0.73, 2.52) | 0.328 | 1.87 | (0.82, 4.25) | 0.135 |
| Smoking | | | | | | |
| Current | 1.00 | | | 1.00 | | |
| Never/Former | 0.74 | (0.50, 1.11) | 0.148 | 0.52 | (0.26, 1.04) | 0.064 |

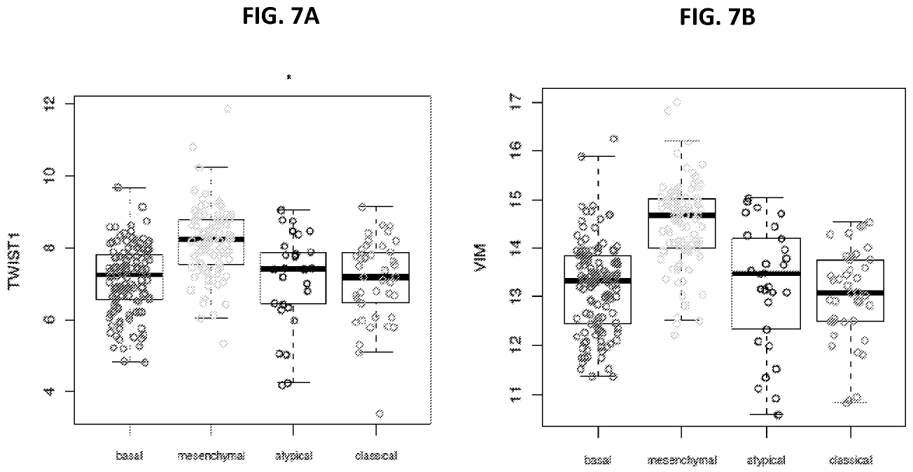

In some embodiments, OCSCC MS subtype is associated with increased expression level of metastasis genes. In some embodiments, the metastasis genes can be associated with the promotion of the epithelial to mesenchymal (EMT) transition. In one embodiment, OCSCC MS subtype has the EMT phenotype. In one embodiment, the EMT phenotype can have significant overexpression of TWIST1 (FIG. 7A). In some embodiments, the OCSCC MS subtype can have at least about 4, at least about 5, at least about 6, at least about 7, at least about 8, at least about 9, at least about 10, at least about 11, or at least about 12, inclusive of all ranges and subranges therebetween, fold increased gene expression levels of TWIST1. In one embodiment, the OCSCC MS subtype can have at least about 8 fold increased gene expression levels of TWIST1. In some embodiments, the OCSCC BA subtype can have at least about 4, at least about 5, at least about 6, at least about 7, at least about 8, at least about 9, or at least about 10, inclusive of all ranges and subranges therebetween, fold increased gene expression levels of TWIST1. In one embodiment, the OCSCC BA subtype can have at least about 7.5 fold increased gene expression levels of TWIST1. In some embodiments, the OCSCC AT subtype can have at least about 4, at least about 5, at least about 6, at least about 7, at least about 8, at least about 9, or at least about 10, inclusive of all ranges and subranges therebetween, fold increased gene expression levels of TWIST1. In one embodiment, the OCSCC AT subtype can have at least about 7.5 fold increased gene expression levels of TWIST1. In some embodiments, the OCSCC CL subtype can have at least about 1, at least about 2, at least about 3, at least about 4, at least about 5, at least about 6, at least about 7, at least about 8, or at least about 9, inclusive of all ranges and subranges therebetween, fold increased gene expression levels of TWIST1. In one embodiment, the OCSCC CL subtype can have at least about 7.5 fold increased gene expression levels of TWIST1.

In another embodiment, the EMT phenotype can have significant overexpression of Vimentin (FIG. 7B). In some embodiments, the OCSCC MS subtype can have at least about 12, at least about 13, at least about 14, at least about 15, at least about 16, at least about 17, at least about 18, at least about 19, or at least about 20, inclusive of all ranges and subranges therebetween, fold increased gene expression levels of Vimentin. In one embodiment, the OCSCC MS subtype can have at least about 15 fold increased gene expression levels of Vimentin. In some embodiments, the OCSCC BA subtype can have at least about 11, at least about 12, at least about 13, at least about 14, at least about 15, at least about 16, or at least about 17, inclusive of all ranges and subranges therebetween, fold increased gene expression levels of Vimentin. In one embodiment, the OCSCC BA subtype can have at least about 13.5 fold increased gene expression levels of Vimentin. In some embodiments, the OCSCC AT subtype can have at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, or at least 16, inclusive of all ranges and subranges therebetween, fold increased gene expression levels of Vimentin. In one embodiment, the OCSCC AT subtype can have at least about 13.5 fold increased gene expression levels of Vimentin. In some embodiments, the OCSCC CL subtype can have at least about 11, at least about 12, at least about 13, at least about 14, or at least about 15, inclusive of all ranges and subranges therebetween, fold increased gene expression levels of Vimentin. In one embodiment, the OCSCC CL subtype can have at least about 13 fold increased gene expression levels of Vimentin.

In some embodiments, the CL subtype can be associated with deregulated oxidative stress pathways. In some embodiments, the CL subtype can be associated with deregulated oxidative stress pathways in any type of HNSCC such as OCSCC and LSCC. In one embodiment, the CL subtype is associated with deregulated oxidative stress pathways in LSCC. In some embodiments, the CL subtype can have mutations in oxidative stress genes. In some embodiments, the oxidative stress gene can be NFE2L2. In some embodiments, the oxidative stress gene can be KEAP1. In some embodiments, the oxidative stress gene can be CUL3. In some embodiments, the CL subtype associated with deregulated oxidative stress pathways can also have TP53 mutations. In some embodiments, the CL subtype associated with deregulated oxidative stress pathways can also have CDKN2A loss-of-function. In some embodiments, the CL subtype associated with deregulated oxidative stress pathways can also have chromosome 3q gains. In some embodiments, the CL subtype associated with deregulated oxidative stress pathways can also have heavy smoking history.

In some embodiments, deregulated oxidative stress pathways can be associated with oncogenesis. In some embodiments, deregulated oxidative stress pathways can be associated with chemo-radiation therapy resistance. In some embodiments, the CL subtype can be associated with chemo-radiation therapy resistance. In some embodiments, the CL subtype can be associated with worse survival.

The present disclosure provides methods for predicting nodal metastasis for a HNSCC patient. In some embodiments, the prediction of nodal metastasis can involve obtaining a head and neck tissue sample for a HNSCC patient. In some embodiments, the HNSCC patients can have various stages of cancers. In some embodiments, the nodal metastasis can be determined by detecting the expression level of at least one subtype classifier of a publically available head and neck cancer database. In some embodiments, a nodal metastasis can be determined by detecting the expression level of any subtype classifiers that are relevant to HNSCC. In one embodiment, the subtype classifiers can be obtained from the TCGA HNSCC gene signature dataset for HNSCC as described herein. In one embodiment, the subtype classifiers can be obtained from the set of 14 subtype classifier (Table 4) relevant to HNSCC as described herein. In one embodiment, the subtype classifiers can be obtained from the Von Walter et al. (PLoS One, 8(2):e56823) gene set as described herein. In one embodiment, the subtype classifiers can be obtained from Table 3 as described herein. In one embodiment, the 14 subtype classifiers can include but are not limited to AKR1C1, NFE2L2, SOX2, KEAP1, RPA2, E2F2, FGFR3, PDGFRA, PDGFRB, TWIST1, EGFR, PIK3CA, TP63, and TGFA (Table 4). In some embodiments, the HNSCC is OCSCC. In some embodiments, the subtyping classifiers can include TP53, RB1, CCND1, and EGFR. In some embodiments, the HNSCC is LSCC. In some embodiments, the HNSCC subject is HPV-negative.

In some embodiments, the MS subtype can be more likely to be associated with nodal metastasis compared with other subtypes such as CL, BA or AT. In some embodiments, the OCSCC MS subtype can be most likely associated with positive lymph node metastasis compared with other OCSCC subtypes such as CL, BA or AT. In some embodiments, the OCSCC MS subtype can be at least about 0.1 times, at least about 0.2 times, at least about 0.3 times, at least about 0.4 times, at least about 0.5 times, at least about 0.6 times, at least about 0.7 times, at least about 0.8 times, at least about 0.9 times, at least about 1 time, at least about 1.2 times, at least about 1.5 times, at least about 1.7 times, at least about 2.0 times, at least about 2.2 times, at least about 2.5 times, at least about 2.7 times, at least about 3.0 times, at least about 3.2 times, at least about 3.5 times, at least about 3.7 times, at least about 4.0 times, at least about 4.2 times, at least about 4.5 times, at least about 4.7 times, at least about 5.0 times, inclusive of all ranges and subranges therebetween, more likely to have occult nodal metastasis compared to other OCSCC subtypes such as CL, BA or AT. In one embodiment, the OCSCC MS subtype can be at least about 3 times more likely to have occult nodal metastasis compared to the BA subtype.

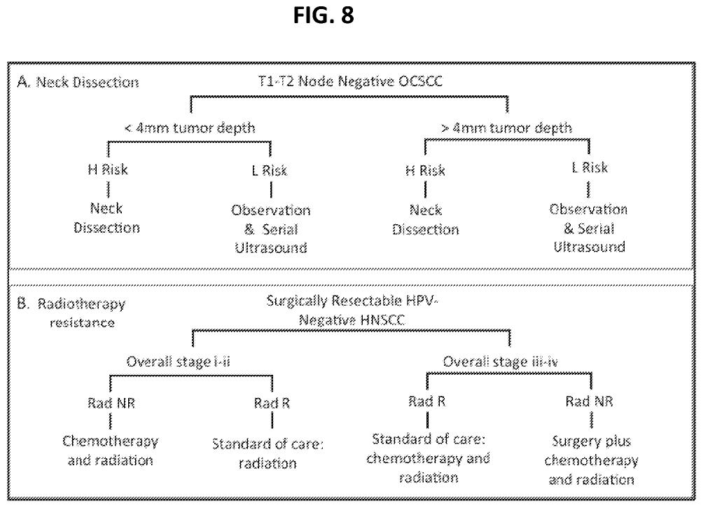

The present disclosure further provides methods for assessing and developing molecular diagnostic assays for clinical applications. For example, as shown in FIG. 8A-8B, clinically and radiographically node-negative OCSCC cases can be assessed for treatment selection by using the gene expression-based diagnostic assay. In one embodiment, OCSCC patients who have less than 4 mm tumor depth and who are associated with high risk MS gene expression as described herein can be stratified to neck dissection treatment. In another embodiment, OCSCC patients who have less than 4 mm tumor depth and who are associated with low risk MS gene expression as described herein can be stratified to observation with serial neck ultrasound detection. In one embodiment, OCSCC patients who have more than 4 mm tumor depth and who are associated with high risk MS gene expression as described herein can be stratified to neck dissection treatment. In another embodiment, OCSCC patients who have more than 4 mm tumor depth and who are associated with low risk MS gene expression as described herein can be stratified to observation with serial neck ultrasound detection.

In some embodiments, the methods for clinical applications as described herein can determine radiotherapy resistance for surgically resectable HPV-negative HNSCC cases. In some embodiments, early stage HPV-negative HNSCC cases such as stage I-II with a low risk gene expression profile can be stratified for radiation therapies. In some embodiments, the low risk gene expression profile can be associated with radiotherapy responder. In some embodiments, the low risk expression profile can be associated with any subtypes except for the CL subtype. In some embodiments, early stage HPV-negative HNSCC cases such as stage I-II with a high risk gene expression profile can be stratified for radiotherapy alone. In some embodiments, early stage HPV-negative HNSCC cases such as stage I-II with a high risk gene expression profile can be stratified for chemotherapy alone. In some embodiments, the high risk expression profile can be associated with the CL subtype. In some embodiments, the high risk expression profile can be associated with radiotherapy non-responder.

In some embodiments, later stage HPV-negative HNSCC cases such as stage III-IV with a low risk gene expression profile can be stratified for radiotherapy. In some embodiments, later stage HPV-negative HNSCC cases such as stage III-IV with a low risk gene expression profile can be stratified for chemotherapies. In some embodiments, the low risk expression profile can be associated with any subtypes except for the CL subtype. In some embodiments, the low risk expression profile can be associated with radiotherapy responder. In some embodiments, later stage HPV-negative HNSCC cases such as stage III-IV with a high risk gene expression profile can be stratified for surgery with radiotherapy. In some embodiments, a high risk gene expression profile can be stratified for surgery with chemotherapy. In some embodiments, a high risk gene expression profile can be stratified for surgery with chemotherapy and radiotherapy. In some embodiments, the high risk expression profile can be associated with the CL subtype. In some embodiments, the high risk expression profile can be associated with radiotherapy non-responder.

### EXAMPLE

The present disclosure is further illustrated by reference to the following Examples. However, it should be noted that these Examples, like the embodiments described above, are illustrative and are not to be construed as restricting the scope of the invention in any way.

### Example 1—Gene Expression Subtype Analysis of Laryngeal and Oral Cavity Squamous Cell Carcinoma Reveals Novel Molecular Markers of Nodal Metastasis and Survival

### Objective

Gene expression analyses of head and neck squamous cell carcinoma have revealed four distinct molecular subtypes: basal, mesenchymal, atypical, and classical. These subtypes show varied mutational and gene expression characteristics and may have predictive or prognostic potential in head and neck cancer.

In this example, a gene expression subtyping analysis of oral cavity and laryngeal squamous cell carcinoma within The Cancer Genome Atlas (TCGA) head and neck cancer cohort² was undertaken. HPV-negative head and neck cancer were deliberately focused on in an attempt to establish novel molecular markers of treatment response and survival for a subset of tumors with persistently poor oncologic outcomes. The aims of this example were 1) to compare the distribution and prognostic significance of gene expression subtypes in oral cavity (OCSCC) and laryngeal (LSCC) squamous cell carcinoma, and 2) to determine the association between gene expression subtype, nodal metastasis, and survival in these groups. It was hypothesized that the distribution of gene expression subtypes will differ between laryngeal and oral cavity squamous cell carcinoma, reflecting different drivers of carcinogenesis in HPV-negative head and neck cancer across anatomic sites. Furthermore, it was hypothesized that gene expression subtypes can be used to predict nodal metastasis and prognosticate survival in head and neck cancer.

### Methods

OCSCC and LSCC cases were identified within the TCGA head and neck cancer dataset. The TCGA² is a comprehensive cancer genomic data repository sponsored by The Cancer Genome Atlas Research Network of the National Cancer Institute, and including DNA sequencing, RNA sequencing, and protein expression data on 33 cancer types. The TCGA head and neck cancer dataset includes 517 cases across all anatomic sites. Clinical, tumor, and treatment data are also available for analysis.² For this analysis, only HPV-negative head and neck cancer were used. Since p16 and HPV status is reported inconsistently in TCGA, oropharyngeal cancers were excluded and this analysis was limited to LSCC and OCSCC.

### RNA Sequencing Analysis

RNA-Seq by Expected Maximization (RSEM)⁴ was used to quantify gene expression levels from TCGA RNA-seq data. The RSEM gene expression measurements for n=517 head and neck cancer cases were transformed using log 2 (RSEM+1) and subsequently median centered by gene, and LSCC (n=125) and OCSCC (n=309) cases were selected for further analysis. The centroids in the gene expression subtype classifier originally presented by Walter et al.¹ (2013) were reduced from 838 genes to 728 genes³ (i.e. Table 3), as described in the TCGA genomic characterization of head and neck cancer cohort.² Each subject was then assigned to one of the four subtypes (basal, mesenchymal, atypical, or classical) by identifying the nearest centroid using a correlation-based similarity metric. A total of 267 of the 279 subjects (95.7%) profiled in the original TCGA head and neck cancer cohort² received the same subtype classification in both analyses.

Gene expression heat maps including the reduced 728 gene see (see Table 3) as well as including 14 genes (i.e., AKR1C1, NFE2L2, SOX2, KEAP1, RPA2, E2F2, FGFR3, PDGFRA, PDGFRB, TWIST1, EGFR, PIK3CA, TP63, and TGFA; see also Table 4) relevant to head and neck squamous cell carcinoma were generated using ConsensusCluster-Plus as described previously^{1, 5} In order to facilitate comparisons between OCSCC and LSCC expression, the 728-gene list (Table 3) was ordered by combining expression data for the OCSCC and LSCC samples, clustering the rows and genes, then retaining the ordering for separate OCSCC and LSCC heat maps. The 14 gene lists (Table 4) were also ordered identically.

### Statistical Analysis

Descriptive statistics were used to describe patient, disease, and treatment characteristics between each gene expression subtype. P-values were calculated with a chi-square test. Overall survival (OS) was measured from baseline diagnosis to death obtained from the National Death Index. Cases were censored at 3 years. Kaplan-Meier curves and log-rank values were calculated. Unadjusted hazard ratios were calculated with Cox proportional hazards model. Proportional hazards assumption was tested and satisfied. Statistical analysis was performed using R version 3.1.4.

Additionally, using gene expression data from TCGA early stage OCSCC cases, whether or not the MS group has an epithelial to mesenchymal transition (EMT) phenotype including significant over-expression of putative EMT drivers TWIST1 was examined. The rates of pathologically positive lymph nodes and survival in 70 T1-T2, clinically node negative OCSCC patients undergoing a tumor resection and a neck dissection was compared by gene expression subtype.

Further, in order to further evaluate whether gene expression subtype and overall survival are prognostic, the association between gene expression subtype and overall survival in TCGA LSCC undergoing primary radiation therapy-based treatment was examined.

### Results

### Descriptive Statistics

First, the distribution and gene expression characteristics of each subtype in the OCSCC and LSCC cohorts are described. Of the 309 OCSCC cases, 128 (41.4%) demonstrated a basal subtype, 103 (33.3%) mesenchymal, 43 (14%) classical, and 35 (11.3%) atypical. Of the 125 LSCC cases, 43 (34.4%) expressed an atypical subtype, 38 (30.4%) classical, 27 (21.6%) mesenchymal, and 12 (9.6%) basal. The demographic, tumor, and treatment characteristics of the OCSCC and LSCC cases by subtype are found in Table 1. There was no significant difference with respect to clinical TNM stage between OCSCC subtypes. Overall, mesenchymal tumors were significantly more likely to be pathologically node positive (65.4% node positive) compared to the other groups. While the classical OCSCC cases were more likely to be smokers, no statistically significant difference is duration or pack year history of tobacco use was noted between the groups. Among LSCC cases, there was no significant difference with respect to race, gender, smoking status, clinical TNM stage, pathologic TNM stage, or adjuvant radiation therapy by gene expression subtypes.

### Gene Expression Profiles

OCSCC and LSCC gene expression heat maps for the 728-gene set are found in FIG. 1A and FIG. 1B, respectively. The 14 gene expression heat-maps for OCSCC and LSCC are found in FIG. 2A and FIG. 2B, respectively. Clustering of cases into the four subtypes based on gene expression signatures among both OCSCC and LSCC cases, with differences in subtype distribution by anatomic site were demonstrated.

For OCSCC (see FIG. 3), basal subtype had the best 3-year survival (62.5%, 95% CI: 54.0%-72.4%) followed by atypical subtype (51.5%, 95% CI: 35.2%-75.2%) and mesenchymal (47.3%, 95% CI: 37.5%-59.8%). Classical subtype had the worst 3-year survival (38.7%, 95% CI: 24.1%-62.1%). For LSCC (see FIG. 5), classical subtype had the worst 3-year survival (43.7%, 95% CI: 30.0-63.7%) and atypical subtype had the best (78.05%, 95% CI: 65.2%-93.2%). Basal and mesenchymal subtypes had similar survival (55.6%, 95% CI: 31.0%-99.7% and 58.3%, 95% CI: 41.1-82.5%, respectively).

The results of a multivariate regression analysis for factors associated with risk of death in OCSCC and LSCC are found in Table II. In OCSCC, gene expression subtype was not statistically associated with an increased risk of death. In LSCC, when compared to the atypical subtype, the classical subtype has an increased risk of death (HR=4.32, 95% CI 1.77-010.54, p=0.001). Although approaching statistical significance, the mesenchymal subtype is also associated with an increased risk of death compared with atypical subtype (HR=2.51, 95% CI 0.91-6.91, p=0.076). Female gender was associated with significantly worse survival compared to male (HR=4.2, 95% CI 1.99-8.90, p<0.001). Also, in LSCC, a significant difference in survival was demonstrated between the CL and AT groups. When limited to LSCC cases undergoing radiation therapy, it was demonstrated that CL and AT comprise the vast majority of cases and that CL was associated with worse survival (CL HR=3.30, 0.89-12.3, p=0.075, FIG. 6).

### Occult Nodal Metastasis in OCSCC

Given the association demonstrated between the OCSCC mesenchymal subtype and nodal metastasis, a subset analysis of T1/T2, clinically node-negative OCSCC cases was conducted in order to test the predictive value of gene expression subtypes in detecting occult nodal metastasis. Of the 67 cases identified that fit criteria for inclusion, 24 (35.8%) expressed a basal subtype, 26 (38.8%) a mesenchymal subtype, 8 (12%) a classical subtype, and 9 (13.4%) an atypical subtype. No significant difference in gender, clinical T-stage, or adjuvant therapy use was noted between the groups. Non-Hispanic Whites were significantly more likely to express a mesenchymal subtype compared to African-Americans and Asians. When risk of occult nodal metastasis was considered, mesenchymal subtype tumors were significantly more likely to have pathologically positive lymph nodes at the time of neck dissection (RR=3.38, 95% CI 1.08-10.69) compared to the other subtypes. Furthermore, the MS group was associated with worse overall survival (HR=3.86, 0.95-16.6, p=0.058, FIG. 4). Under current standard practices, the rate of positive lymph nodes in patients undergoing elective neck dissection is 20%, with 80% undergoing unnecessary neck surgery. Also, the MS group had an epithelial to mesenchymal transition (EMT) phenotype including significant over-expression of putative EMT drivers TWIST1 (FIG. 7A) and Vimentin (FIG. 7B).

### Conclusions

Substantive differences in the distribution of gene expression patterns in OCSCC and LSCC were demonstrated. OCSCC cases were comprised primarily of the mesenchymal and basal subtypes, while LSCC was comprised primarily of classical and atypical subtypes. In OCSCC, the mesenchymal subtype, characterized by epithelial to mesenchymal transition expression, was significantly associated with nodal metastasis. In a subset analysis of clinically T1-2N0M0 OCSCC, the mesenchymal subtype was demonstrated to be predictive of occult nodal metastasis (RR=3.38, 95% CI 1.08-10.69). In LSCC, the classical subtype, characterized by KEAP1/NRF2 pathway alterations, was associated with significantly worse overall survival (HR=4.32, 95% CI 1.77-10.54, p=0.001).

This analysis of gene expression subtypes in OCSCC and LCSCC revealed potential novel markers of nodal metastasis and survival in HPV-negative head and neck cancer, and highlights the biologic heterogeneity of this disease. Future studies will continue to refine and validate these gene expression subtypes, with the goal of providing molecular risk assessments that guide therapy and improve patient outcomes.

### INCORPORATION BY REFERENCE

The following references are incorporated by reference in their entireties for all purposes.
1. Walter V, Yin X, Wilkerson M D, et al. Molecular subtypes in head and neck cancer exhibit distinct patterns of chromosomal gain and loss of canonical cancer genes. PloS one. 2013; 8(2):e56823.
2. Cancer Genome Atlas N. Comprehensive genomic characterization of head and neck squamous cell carcinomas. Nature. Jan. 29 2015; 517(7536):576-582.
3. Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.
4. Li B, Dewey C N. RSEM: accurate transcript quantification from RNA-Seq data with or without a reference genome. BMC Bioinformatics. Aug. 4 2011; 12:323.
5. Wilkerson M D, Hayes D N. ConsensusClusterPlus: a class discovery tool with confidence assessments and item tracking. Bioinformatics. Jun. 15 2010; 26(12):1572-1573.
6. Siegel R L, Miller K D, Jemal A. Cancer Statistics, 2015. CA Cancer J Clin. 2015; 65: 5-29. doi:10.3322/caac.21254
7. Lawrence M S, Sougnez C, Lichtenstein L, Cibulskis K, Lander E, Gabriel S B, et al. Comprehensive genomic characterization of head and neck squamous cell carcinomas. Nature. 2015; 517: 576-582. doi:10.1038/nature14129
8. Wichman G, Rosolowski M, Krohn K, et al. The role of HPV RNA transcription, immune response-related gene expression and disruptive TP53 mutations in diagnostic and prognostic profiling of head and neck cancer. Intl Jrnl Cancer 2015; 137: 2846-2857.
9. Keck M K, Zuo Z, Khattri a., Stricker T P, Brown C D, Imanguli M, et al. Integrative Analysis of Head and Neck Cancer Identifies Two Biologically Distinct HPV and Three Non-HPV Subtypes. Clin Cancer Res. 2014; 21: 870-881. doi:10.1158/1078-0432.CCR-14-2481
10. Dabney A R. ClaNC: Point-and-click software for classifying microarrays to nearest centroids. Bioinformatics. 2006; 22: 122-123. doi:10.1093/bioinformatics/bti756
11. Tibshirani R, Hastie T, Narasimhan B, Chu G. Diagnosis of multiple cancer types by shrunken centroids of gene expression. Proc Natl Acad Sci USA. 2002; 99: 6567-72. doi:10.1073/pnas.082099299
12. Bindea G, Mlecnik B, Tosolini M, et al. Spatiotemporal dynamics of intratumoral immune cells reveal the immune landscape in human cancer. Immunity 2013; 39(4):782-95.
13. Chung C H, Parker J S, Karaca G, et al. Molecular classification of head and neck squamous cell carcinomas using patterns of gene expression. Cancer cell. May 2004; 5(5):489-500.

### Example 2—Development of Prognostic Marker of Occult Nodal Metastases for OCSCC Patients

### Objective

This example will be performed to develop a prognostic assay for detecting and assessing the risks and likelihood of occult nodal metastases in early-stage, node-negative OCSCC using subtype gene expression, tumor mutations, and clinical features. The objective was also to inform the need for performing neck surgeries in OCSCC patients. This example will be a follow-up and validation of the analyses conducted in Example 1.

### Methods

Residual archived FFPE tissue from 200 oral cavity clinical tumor samples will be collected from the University of North Carolina archive for gene expression RNAseq and DNA sequencing. Tissues will be derived from oral cavity cancer patients treated between 2008 and 2013. Patients will be stratified into two groups: (1) T1-T2 N0M0 oral cavity cancer undergoing neck dissection and pathologically N0, and (2) T1-T2, clinically N0M0 oral cavity cancers undergoing neck dissection that are pathologically node-positive. Survival and recurrence data will be collected for each patient through a systematic chart review by a trained medical abstractor. HPV negative OCSCC tumors will be confirmed using E6/E7 gene expression already built into the subtyping assay. Targeted DNAseq for ˜50 genes including TP53, RB1, CCND1, EGFR and post sequencing data processing will be performed on all 200 OCSCC samples. DNA will be extracted from macrodissected tissues using the Promega-Maxwell automated nucleic acid extraction system and quantified by OD260/280 ratios using PicoGreen. Libraries will be constructed using Agilent Sure Select custom targeted exome kits with 200 ng DNA input and QC'd using the Illumina MiSeq system. DNAseq will be performed using the Illumina HiSeq 4000 platform with a 2×100 bp configuration and 500× average coverage data for each sample will be generated. Sequence data will be QC'd using FastQC and aligned against reference genome hg19 using BWA. SNV's and indels will be called using open source tools, namely GATK, UNCseqR, and ABRA. Germline and somatic variants will be annotated using dbSNP and Cosmic databases. Mutation data generated by DNAseq, together with the gene expression subtype and clinical history data will be used to develop a prognostic model for use in FFPE tissues to inform decisions regarding elective versus therapeutic neck dissection in OCSCC patients.

The primary performance criteria for this assay will be the ability to predict nodal metastasis in early stage, clinical and radiographically node-negative OCSCC. The nearest centroid predictor from Example 1 (i.e., 728 gene signature classifier; Table 3) will be integrated with clinical features including smoking status, age, tumor size and node status and molecular markers including P53 mutation, CCND1 amplification, RB1 loss and EGFR mutation to provide a prognostic assay. This integrated assay will be evaluated for improved prognostic prediction performance over subtyping alone with respect to prognosticating risk of nodal metastasis. Elastic Net methods that perform both variable selection from multiple data types and parameter estimation (R package—glmnet) will be applied to integrate gene expression data, mutation data, copy number variants, and clinical-pathological variables to improve models for overall survival [1]. Rather than treating cancer subtype as a categorical variable, subtype centroid correlations will be included as variables in the predictors. C-index [2] will be assessed using the models with subtype alone and in combination with clinical features [3] and molecular predictors. Previous research suggests that 20% of early stage, clinically and radiographically node-negative OCSCC will have occult nodal metastasis. Preliminary data suggested that approximately 30% OCSCC cases are MS and 66% are BA gene expression subtypes. If the relative risk of nodal metastasis is assumed to be 2.5 times higher in the MS compared to the BA subtype, the number of samples needed to demonstrate this association is 162. Therefore, 200 cases will be sufficient to support the hypothesis.

### Expected Results:

A power calculation suggests that 162 OCSCC samples should be sufficient to identify statistically significant prognostic differences as described above. If statistical significance is not reached with 200 samples, but data demonstrates a trend in the right direction, more samples to can be collected to reach statistical significance.

### INCORPORATION BY REFERENCE

The following references are incorporated by reference in their entireties for all purposes.
1. Hoadley K A, Yau C, Wolf D M, Cherniack A D, Tamborero D, Ng S, et al. Multiplatform Analysis of 12 Cancer Types Reveals Molecular Classification within and across Tissues of Origin. Cell. Elsevier Inc.; 2014; 1-16. doi:10.1016/j.cell.2014.06.049
2. Harrell F E, Lee K L, Mark D B. Multivariable prognostic models: issues in developing models, evaluating assumptions and adequacy, and measuring and reducing errors. 1996; 15: 361-387.
3. Parker J S, Mullins M, Cheang M C U, Leung S, Voduc D, Vickery T, et al. Supervised risk predictor of breast cancer based on intrinsic subtypes. J Clin Oncol. 2009; 27: 1160-7. doi:10.1200/JCO.2008.18.1370

### Example 3—Development of Assays for Predicting a Radiotherapy Response for HPV-Negative LSCC Patients

### Objective

This example will be performed to develop diagnostic assays for defining radiotherapy treatment responders and non-responders, and therefore, specifically predicting the likelihood for radiotherapy resistance using subtype gene expression, tumor mutations, and clinical features. The integrated diagnostic assay will incorporate gene expression, clinical, and other molecular factors and will be optimized for radiotherapy predictive applications. The objective of this example also includes identifying the radiotherapy resistance populations and informing the need for receiving alternative treatment regimens. This example will be a follow-up and validation of the analyses conducted in Example 1 and will utilize the 728 gene signature sub-typer (Table 3) described in Example 1. To develop the assay, one-hundred-fifty (150) patients with HPV-negative tumors of the larynx receiving primary radiation-based treatment will be identified from the UNC tumor registry and stratified by treatment response.

### Methods

To identify the subtype classifiers of LSCC, the subtype classifier gene expression analyses as described in Example 1 will be used. More specifically, about 200 FFPE stage I and II HPV-negative larynx and/or oropharynx and/or hypopharynx cancer samples from the UNC Translational Pathology Laboratory (TPL) under an IRB-approved protocol will be collected and used for conducting RNAseq and DNAseq analyses as described in Example 2 including the 728 gene panel (Table 3) for RNAseq analysis and the about 50 gene targeted DNAseq panel including TP53, RB1, CCND1, EGFR and post sequencing data processing.

More specifically, elastic net methods as described in Example 2 will be performed to evaluate the integration of clinical features and molecular markers in the development of an assay to predict radiotherapy response in HPV-negative HNSCC tumors. The integration of data, including the mutation of genes implicated in radiotherapy resistance, (NFE2L2, KEAP1 and CUL3) as well as clinical features including tumor size, nodal status and age will be evaluated for enhanced radiotherapy predictive model performance. Performance evaluation will be centered on the ability of the assay to guide decision-making regarding surgical intervention versus radiotherapy alone for HPV-negative HNSCC.

### Expected Results:

A power calculation suggests that 165 HPV-negative laryngeal tumor samples are needed to achieve 80% power to detect a significant difference between the locoregional response rate in the classical subtype, which comprises 21% of HPVnegative HNSCC [1], versus that in all other subtypes. Assumptions used for this calculation include a 5-year 50% locoregional response rate in HPV-negative tumors [2] and a 30% rate in the classical subtype.

It is possible that biopsy sample size and availability may be limited for larynx tumors since tumors treated with radiation therapy will be assessed and not surgically resected. However, this issue can be mitigated since the reduced 728 gene assay will be used and full transcriptome sequencing will not be necessary to subtype tumors, lessening template input requirements. Furthermore, if sufficient material cannot be obtained from the early stage biopsies, recurrent surgical samples may be used provided some additional experiments to demonstrate that subtype is stable and consistent between early stage tumors and post radiotherapy recurrence tumors. Alternatively, investigators at other sites in North Carolina may be recruited to the study to increase the available samples.

### INCORPORATION BY REFERENCE

The following references are incorporated by reference in their entireties for all purposes.
1. Walter V, Yin X, Wilkerson M D, Cabanski C R, Zhao N, Du Y, et al. Molecular subtypes in head and neck cancer exhibit distinct patterns of chromosomal gain and loss of canonical cancer genes. PLoS One. 2013:8:e56823.
2. Lassen P, Primdahl H, Johansen J, Kristensen C A, Andersen E, Andersen U, et al. Impact of HPVassociated p16-expression on radiotherapy outcome in advanced oropharynx and non-oropharynx cancer q. Radiother Oncol. Elsevier Ireland Ltd; 2014; 113: 310-316.

### Example 4—Establishment of a Cost-Effective Assay for Clinical Applications Objective

This example will demonstrate the use of assays from Examples 2 and 3 in the clinical management of head and neck cancer, and as drug development stratification tools supporting more efficient identification of responder population defined by biologic subtypes.

### Methods

To use the assays for clinical applications, multi-institutional prospective clinical trials using gene expression subtyping to direct therapy and management will be implemented. Potential clinical trials based on the two clinical scenarios outlined in this proposal are outlined in FIG. 8A and FIG. 8B. T1-T2, clinically and radiographically node-negative OCSCC cases will undergo gene expression-based diagnostic assay at the time of diagnosis. Patients with tumors <4 mm of invasion and a low-risk non-mesenchymal gene expression profile will be observed, while those with a high-risk mesenchymal gene expression profile will be stratified to neck dissection versus observation with serial neck ultrasounds. Patients with tumors >4 mm invasion and low risk non-mesenchymal gene expression profile will be stratified to neck dissection versus observation with serial neck ultrasounds, while those with >4 mm invasion and a high-risk mesenchymal gene expression profile will undergo neck dissection.

Study endpoints will include nodal metastasis, recurrence or death. Treatment escalation for HPV-negative HNSCC based on gene expression profile: Early stage HPV-negative cancers (T1-T2N0, overall stage I-II) with a low-risk non-classical gene expression profile will be treated with standard of care radiation therapy, while those with a high-risk classical gene expression profile will be stratified to radiation alone versus concurrent chemoradiation. Surgically resectable, HPV-negative overall stage III/IV HNSCC cases will undergo gene expression subtyping at the time of diagnosis. High-risk classical subtype tumors will be stratified into standard of care concurrent chemoradiation versus primary surgical resection and adjuvant chemoradiation. Study endpoints will include recurrence or death.

### Results

The results of the proposed treatment management for the HNSCC patient samples evaluated by the present novel molecular subtyping assays will be monitored for accuracy and efficacy.

### INCORPORATION BY REFERENCE

The following references are incorporated by reference in their entireties for all purposes.
Zevallos et al., Gene Expression Subtype Analysis of Laryngeal and Oral Cavity Squamous Cell Carcinoma reveals Novel Molecular Markers of Nodal Metastasis and Survival. Submitted as Thesis to Triological Society. 2017.
Siegel R L, Miller K D, Jemal A. Cancer Statistics, 2015. CA Cancer J Clin. 2015; 65: 5-29. doi:10.3322/caac.21254
Lawrence M S, Sougnez C, Lichtenstein L, Cibulskis K, Lander E, Gabriel S B, et al. Comprehensive genomic characterization of head and neck squamous cell carcinomas. Nature. 2015; 517: 576-582. doi:10.1038/nature14129
Von Walter, Yin X, Wilkerson M D, Cabanski C R, Zhao N, Du Y, Ang M K, Hayward M C, Salazar A H, Hoadley K A, Fritchie K, Sailey C J, Weissler M C, Shockley W W, Zanation A M, Hackman T, Thorne L B, Funkhouser W D, Muldrew K L, Olshan A F, Randell S H, Wright F A, Shores C G, Hayes D N. (2013). Molecular Subtypes in Head and Neck Cancer Exhibit Distinct Patterns of Chromosomal Gain and Loss of Canonical Cancer Genes. PLoS One, 8(2):e56823. PMCID: 3579892.
Wichman G, Rosolowski M, Krohn K, et al. The role of HPV RNA transcription, immune response-related gene expression and disruptive TP53 mutations in diagnostic and prognostic profiling of head and neck cancer. Intl Jrnl Cancer 2015; 137: 2846-2857.
Keck M K, Zuo Z, Khattri a., Stricker T P, Brown C D, Imanguli M, et al. Integrative Analysis of Head and Neck Cancer Identifies Two Biologically Distinct HPV and Three Non-HPV Subtypes. Clin Cancer Res. 2014; 21: 870-881. doi:10.1158/1078-0432.CCR-14-2481
Dabney A R. ClaNC: Point-and-click software for classifying microarrays to nearest centroids. Bioinformatics. 2006; 22: 122-123. doi:10.1093/bioinformatics/bti756
Tibshirani R, Hastie T, Narasimhan B, Chu G. Diagnosis of multiple cancer types by shrunken centroids of gene expression. Proc Natl Acad Sci USA. 2002; 99: 6567-72. doi:10.1073/pnas.082099299
Bindea G, Mlecnik B, Tosolini M, et al. Spatiotemporal dynamics of intratumoral immune cells reveal the immune landscape in human cancer. Immunity 2013; 39(4):782-95.
Chung C H, Parker J S, Karaca G, et al. Molecular classification of head and neck squamous cell carcinomas using patterns of gene expression. Cancer cell. May 2004; 5(5):489-500.

TABLE 3
728 Classifier Biomarkers For HNSCC Subtypes

| | | GenBank |
|---|---|---|
| Number | Gene Symbol | Accession No.* |
| 1. | ARMCX3 | NM_016607.3 |
| 2. | AKAP7 | NM_004842.3 |
| 3. | GSPT2 | NM_018094.4 |
| 4. | DNMT3A | NM_175629.2 |
| 5. | LTBP3 | NM_001130144.2 |
| 6. | CAND2 | NM_001162499.1 |
| 7. | SMARCD3 | NM_001003802.1 |
| 8. | C3orf18 | NM_016210.4 |
| 9. | PRKD1 | NM_001330069.1 |
| 10. | DPYSL3 | NM_001197294.1 |
| 11. | GPRC5B | NM_016235.2 |
| 12. | LRIG1 | NM_015541.2 |
| 13. | TTC28 | NM_001145418.1 |
| 14. | PATZ1 | NM_014323.2 |
| 15. | VEZF1 | NM_007146.2 |
| 16. | DISP1 | NM_032890.4 |
| 17. | PDE4A | NM_001111307.1 |
| 18. | SCAMP5 | NM_001178111.1 |
| 19. | ARNT2 | NM_014862.3 |
| 20. | BRP44L | AF181116.1 |
| 21. | C21orf33 | NM_004649.7 |
| 22. | ABCA7 | NM_019112.3 |
| 23. | ECHDC2 | NM_001198961.1 |
| 24. | SLC37A1 | NM_001320537.1 |
| 25. | TMCO4 | NM_181719.5 |
| 26. | FAAH2 | NM_174912.3 |
| 27. | SLC25A23 | NM_024103.2 |
| 28. | C22orf32 | NM_033318.4 |
| 29. | FAM117A | NM_030802.3 |
| 30. | CLU | NM_001831.3 |
| 31. | SCARA3 | NM_016240.2 |
| 32. | CBFA2T3 | NM_005187.5 |
| 33. | RAB37 | NM_001006638.2 |
| 34. | FAM171A1 | NM_001010924.1 |
| 35. | SETMAR | NM_006515.3 |
| 36. | TNRC6C | NM_001142640.1 |
| 37. | CHPT1 | NM_020244.2 |
| 38. | GKAP1 | NM_025211.3 |
| 39. | PHF17 | BC032376.1 |
| 40. | PLCL2 | NM_001144382.1 |
| 41. | FOXA1 | NM_004496.3 |
| 42. | EYA2 | NM_005244.4 |
| 43. | GALNT12 | NM_024642.4 |
| 44. | HLF | NM_002126.4 |
| 45. | LMO4 | NM_006769.3 |
| 46. | PLAC8 | NM_001130716.1 |
| 47. | KATNAL2 | NM_001353899.1 |
| 48. | ICA1 | NM_022307.2 |
| 49. | CREB3L4 | NM_130898.3 |
| 50. | MYO5C | NM_018728.3 |
| 51. | ZSCAN16 | NM_025231.2 |
| 52. | MEIS1 | NM_002398.2 |
| 53. | SYTL4 | NM_080737.2 |
| 54. | JUP | NM_002230.3 |
| 55. | GCHFR | NM_005258.2 |
| 56. | TCEA3 | NM_003196.2 |
| 57. | GLS2 | NM_013267.3 |
| 58. | MYB | NM_001130173.1 |
| 59. | P4HTM | NM_177939.2 |
| 60. | PARD6A | NM_016948.2 |
| 61. | Clorf115 | NM_024709.4 |
| 62. | SYNGR1 | NM_004711.4 |
| 63. | MAP2K6 | NM_002758.3 |
| 64. | PBX1 | NM_002585 |
| 65. | REPIN1 | NM_013400.3 |
| 66. | PPARG | NM_005037.5 |
| 67. | KRT15 | NM_002275.3 |
| 68. | ANXA11 | NM_001157.2 |
| 69. | AKR1A1 | NM_006066.3 |
| 70. | C2orf55 | NM_207362.2 |
| 71. | BLNK | NM_013314.3 |
| 72. | DOPEY2 | NM_001320714.1 |
| 73. | DENND2D | NM_024901.4 |
| 74. | ARHGEF10L | NM_018125.3 |
| 75. | SASH1 | NM_015278.4 |
| 76. | ABR | NM_021962.4 |
| 77. | CCDC12 | NM_144716.5 |
| 78. | FMO2 | NM_001460.4 |
| 79. | IKZF2 | NM_016260 |
| 80. | HPGD | NM_001256307.1 |
| 81. | GPD1L | NM_015141.3 |
| 82. | SH3BGRL2 | NM_031469.3 |
| 83. | ZNF132 | NM_003433.3 |
| 84. | BCAS1 | NM_003657.3 |
| 85. | TJP3 | NM_001267560 |
| 86. | PSCA | NM_005672.4 |
| 87. | TSPAN6 | NM_003270.3 |
| 88. | SORBS2 | NM_003603.6 |
| 89. | FUT7 | NM_004479.3 |
| 90. | TNFRSF10C | NM_003841.3 |
| 91. | ESPL1 | NM_012291.4 |
| 92. | RECQL5 | NM_004259.6 |
| 93. | ACADSB | NM_001609.3 |
| 94. | SLC9A3R1 | NM_004252 |
| 95. | AGFG2 | NM_006076.4 |
| 96. | CLDN7 | NM_001307.5 |
| 97. | ELF3 | NM_004433 |
| 98. | MUC20 | NM_001282506 |
| 99. | SCNN1A | NM_001038 |
| 100. | CEACAM1 | NM_001712.4 |
| 101. | MUC4 | NM_018406 |
| 102. | EPHX2 | NM_001979.5 |
| 103. | ST6GALNAC1 | NM_018414.4 |
| 104. | CSTB | NM_000100 |
| 105. | MAL2 | NM_052886 |
| 106. | CSTA | NM_005213 |
| 107. | GRHL3 | NM_021180 |
| 108. | RAB25 | NM_020387 |
| 109. | C2orf54 | NM_001085437.2 |
| 110. | PPL | NM_002705 |
| 111. | SPINK5 | NM_001127698 |
| 112. | GBP6 | NM_198460.2 |
| 113. | PITX1 | NM_002653.4 |
| 114. | NMU | NM_006681.3 |
| 115. | ANXA9 | NM_003568.2 |
| 116. | CEACAM5 | NM_004363 |
| 117. | PRSS27 | NM_031948 |
| 118. | TTC9 | NM_015351 |
| 119. | SMAGP | NM_001031628.1 |
| 120. | CLCA4 | NM_012128.3 |
| 121. | MAL | NM_002371.3 |
| 122. | RMND5B | NM_001288794.1 |
| 123. | RBM47 | NM_001098634.1 |
| 124. | SH2D4A | NM_022071 |
| 125. | MACC1 | NM_182762.3 |
| 126. | USP6NL | NM_014688.3 |
| 127. | FBXO34 | NM_017943.3 |
| 128. | TACSTD2 | NM_002353.2 |
| 129. | ALDH9A1 | NM_000696.3 |
| 130. | MGST2 | NM_002413.4 |
| 131. | GPT2 | NM_133443.3 |
| 132. | WIBG | NM_001046823.1 |
| 133. | MANSC1 | NM_018050.3 |
| 134. | TMPRSS11A | NM_182606 |
| 135. | TLR5 | NM_003268.5 |
| 136. | CA13 | NM_198584.2 |
| 137. | PLEKHA7 | NM_001329630.1 |
| 138. | CCDC56 | NM_206340.4 |
| 139. | RTKN2 | NM_145307.3 |
| 140. | TIFA | NM_052864.2 |
| 141. | KLF5 | NM_001730 |
| 142. | NACA2 | NM_199290.3 |
| 143. | CD302 | NM_014880.4 |
| 144. | CREB5 | NM_001011666.2 |
| 145. | TMEM158 | NM_015444.2 |
| 146. | EGLN1 | NM_022051.2 |
| 147. | FAM36A | NM_198076.5 |
| 148. | SIPA1L2 | NM_020808.4 |
| 149. | ATP2B1 | NM_001001323.1 |
| 150. | ANKRD40 | NM_052855.3 |
| 151. | EIF2C2 | NM_012154.4 |
| 152. | NLN | NM_020726.4 |
| 153. | B4GALNT1 | NM_001478.4 |
| 154. | DGKG | NM_001346.2 |
| 155. | PFN2 | NM_053024.3 |
| 156. | LRP12 | NM_013437.4 |
| 157. | SEMA4F | NM_004263.4 |
| 158. | XPR1 | NM_004736.3 |
| 159. | SMYD3 | NM_001167740.1 |
| 160. | ORC6L | NM_014321.3 |
| 161. | FAM119A | NM_145280.6 |
| 162. | HOXA10 | NM_018951.3 |
| 163. | COCH | NM_001135058.1 |
| 164. | RAD51L3 | NM_002878.3 |
| 165. | ABI2 | NM_001282925.1 |
| 166. | RAB6B | NM_016577 |
| 167. | KCNMB3 | NM_171828.2 |
| 168. | C1orf31 | NM_001012985.2 |
| 169. | CABYR | NM_012189 |
| 170. | ABCC1 | NM_004996 |
| 171. | AKR1C1 | NM_001353 |
| 172. | SRXN1 | NM_080725.2 |
| 173. | TXNRD1 | NM_182729 |
| 174. | ADAM23 | NM_003812.3 |
| 175. | GPX2 | NM_002083.3 |
| 176. | PIR | NM_003662 |
| 177. | SLCO3A1 | NM_013272.3 |
| 178. | CASK | NM_003688.3 |
| 179. | KCNK1 | NM_002245.3 |
| 180. | TMEM14A | NM_014051.3 |
| 181. | ZFP64 | NM_018197.2 |
| 182. | ETV5 | NM_004454.2 |
| 183. | PTCH1 | NM_001083602.2 |
| 184. | CHST7 | NM_019886 |
| 185. | C6orf168 | NM_032511 |
| 186. | FZD7 | NM_003507.1 |
| 187. | HEY1 | NM_001282851.1 |
| 188. | HHAT | NM_018194.5 |
| 189. | NTRK2 | NM_006180 |
| 190. | ALDH1A1 | NM_000689.4 |
| 191. | GCNT2 | NM_145649 |
| 192. | PRKX | NM_005044 |
| 193. | SLC16A14 | NM_152527 |
| 194. | LONRF1 | NM_152271.4 |
| 195. | ZDHHC2 | NM_016353 |
| 196. | MARCH9 | NM_138396.5 |
| 197. | RASSF9 | NM_005447.3 |
| 198. | EPCAM | NM_002354 |
| 199. | UCHL1 | NM_004181.4 |
| 200. | TNKS | NM_003747.2 |
| 201. | DSG2 | NM_001943.4 |
| 202. | C12orf47 | NR_015404.1 |
| 203. | KLHL12 | NM_001303051.1 |
| 204. | DHX9 | NM_001357.4 |
| 205. | ILF2 | NM_004515.3 |
| 206. | FAM72A | NM_001123168.2 |
| 207. | NEK2 | NM_002497.3 |
| 208. | UBE2T | NM_014176.4 |
| 209. | CLCN2 | NM_004366 |
| 210. | RCN2 | NM_002902.2 |
| 211. | FAM20B | NM_014864.3 |
| 212. | RFWD2 | NM_022457.6 |
| 213. | DLG1 | NM_001098424.1 |
| 214. | ABCC5 | NM_005688 |
| 215. | RNF7 | NM_014245.4 |
| 216. | NCBP2 | NM_007362.3 |
| 217. | SENP2 | NM_021627.2 |
| 218. | ACTL6A | NM_004301.4 |
| 219. | ZNF639 | NM_016331.2 |
| 220. | MYNN | NM_018657.4 |
| 221. | PHC3 | NM_001308116.1 |
| 222. | LSG1 | NM_018385.2 |
| 223. | RSRC1 | NM_001271838.1 |
| 224. | TMEM97 | NM_014573.2 |
| 225. | TMPO | NM_003276.2 |
| 226. | MRPL55 | NM_181454.2 |
| 227. | TOMM20 | NM_014765.2 |
| 228. | RPS6KC1 | NM_012424.5 |
| 229. | MEST | NM_002402.3 |
| 230. | WDR67 | NM_145647.3 |
| 231. | TERF1 | NM_017489.2 |
| 232. | SOX12 | NM_006943.3 |
| 233. | C19orf2 | NM_003796.3 |
| 234. | HSPB2 | NM_001541.3 |
| 235. | APOLD1 | NM_001130415.1 |
| 236. | AG2 | NM_001305068.1 |
| 237. | NR4A3 | NM_006981.3 |
| 238. | SUSD2 | NM_019601.3 |
| 239. | FBLN2 | NM_001004019.1 |
| 240. | DLC1 | NM_182643.2 |
| 241. | FGFR1 | NM_023110.2 |
| 242. | LOXL3 | NM_032603.4 |
| 243. | ADAM12 | NM_003474.5 |
| 244. | FERMT2 | NM_006832.2 |
| 245. | DACT1 | NM_016651.5 |
| 246. | FN1 | NM_001306131.1 |
| 247. | CTGF | NM_001901.2 |
| 248. | ADAMTS2 | NM_014244.4 |
| 249. | COL3A1 | NM_000090.3 |
| 250. | PDGFRB | NM_002609.3 |
| 251. | COL5A1 | NM_000093.4 |
| 252. | COL5A2 | NM_000393.4 |
| 253. | COL1A2 | NM_000089.3 |
| 254. | COL6A1 | NM_001848 |
| 255. | COL6A2 | NM_001849 |
| 256. | COL6A3 | NM_004369.3 |
| 257. | SPARC | NM_003118 |
| 258. | GLT8D2 | NM_031302.4 |
| 259. | OLFML2B | NM_001297713 |
| 260. | CHN1 | NM_001822.5 |
| 261. | LAMA4 | NM_001105206.2 |
| 262. | NID2 | NM_007361.3 |
| 263. | ITGA1 | NM_181501.1 |
| 264. | THBS2 | NM_003247.3 |
| 265. | COL8A1 | NM_001850.4 |
| 266. | FSTL1 | NM_007085.4 |
| 267. | VCAN | NM_004385.4 |
| 268. | PMP22 | NM_000304 |
| 269. | COL12A1 | NM_004370.5 |
| 270. | CALD1 | NM_033138 |
| 271. | MICAL2 | NM_001282663.1 |
| 272. | LEPRE1 | NM_022356 |
| 273. | LOXL2 | NM_002318.2 |
| 274. | SERPINH1 | NM_001207014 |
| 275. | CHSY1 | NM_014918.4 |
| 276. | TWIST1 | NM_000474.3 |
| 277. | CRISPLD2 | NM_031476.3 |
| 278. | BGN | NM_001711.5 |
| 279. | RCN3 | NM_020650 |
| 280. | GLIS3 | NM_001042413.1 |
| 281. | FNDC1 | NM_032532.2 |
| 282. | P4HA3 | NM_182904.4 |
| 283. | ACTA2 | NM_001141945.2 |
| 284. | MYL9 | NM_006097.4 |
| 285. | TAGLN | NM_001001522 |
| 286. | LMOD1 | NM_012134.2 |
| 287. | CLEC11A | NM_002975.2 |
| 288. | HEYL | NM_014571.3 |
| 289. | EMILIN1 | NM_007046.3 |
| 290. | THY1 | NM_006288.4 |
| 291. | GGT5 | NM_001099781.2 |
| 292. | LRRC32 | NM_005512.2 |
| 293. | MFRP | NM_031433.3 |
| 294. | BNC2 | NM_017637.5 |
| 295. | FBXL7 | NM_012304.4 |
| 296. | EBF1 | NM_001290360.2 |
| 297. | CNRIP1 | NM_015463.2 |
| 298. | GPR124 | NM_032777.9 |
| 299. | GNG11 | NM_004126.3 |
| 300. | LHFP | NM_175386.3 |
| 301. | ATP10A | NM_024490.3 |
| 302. | DCHS1 | NM_003737.3 |
| 303. | FAM198B | NM_001031700.2 |
| 304. | AEBP1 | NM_001129.4 |
| 305. | PCOLCE | NM_002593 |
| 306. | CTSK | NM_000396.3 |
| 307. | MXRA8 | NM_001282585.1 |
| 308. | RARRES2 | NM_002889.3 |
| 309. | OLFML3 | NM_020190 |
| 310. | TGFB3 | NM_003239 |
| 311. | COLEC12 | NM_130386.2 |
| 312. | ZNF521 | NM_015461.2 |
| 313. | RECK | NM_021111.2 |
| 314. | DACT3 | NM_145056.2 |
| 315. | ANGPTL2 | NM_012098.2 |
| 316. | IGDCC4 | NM_020962.2 |
| 317. | PPAPDC3 | NM_032728.3 |
| 318. | PTH1R | NM_000316 |
| 319. | ZCCHC24 | NM_153367.3 |
| 320. | DCN | NM_001920.4 |
| 321. | SPON1 | NM_006108.3 |
| 322. | SFRP2 | NM_003013.2 |
| 323. | SFRP4 | NM_003014.3 |
| 324. | VGLL3 | NM_016206.3 |
| 325. | FBN1 | NM_000138.4 |
| 326. | RAB3IL1 | NM_013401.3 |
| 327. | C1orf54 | NM_001301039.1 |
| 328. | DAB2 | NM_001343.3 |
| 329. | ZEB2 | NM_014795.3 |
| 330. | PALM2- | NM_007203.4 |
| | AKAP2 | |
| 331. | NTNG2 | NM_032536.3 |
| 332. | MRAS | NM_012219.4 |
| 333. | NNMT | NM_006169 |
| 334. | PKIG | NM_181805.2 |
| 335. | AMPD2 | NM_004037.7 |
| 336. | FAM176B | NM_018166.2 |
| 337. | FAM20C | NM_020223.3 |
| 338. | LAMB2 | NM_002292.3 |
| 339. | RGS3 | NM_130795.3 |
| 340. | IGFBP7 | NM_001553.2 |
| 341. | SLC39A13 | NM_001128225.2 |
| 342. | TPM2 | NM_213674.1 |
| 343. | TIMP1 | NM_003254.2 |
| 344. | PGM2L1 | NM_173582.4 |
| 345. | CMTM3 | NM_144601 |
| 346. | C1orf216 | NM_152374.1 |
| 347. | FLJ10357 | BC142692.1 |
| 348. | HTRA3 | NM_053044.4 |
| 349. | MARVELD1 | NM_031484.3 |
| 350. | MFAP2 | NM_017459.2 |
| 351. | PHLDB1 | NM_015157 |
| 352. | TPM1 | NM_001018005.1 |
| 353. | GPX8 | NM_001008397 |
| 354. | PARVA | NM_018222.5 |
| 355. | CPXM1 | NM_019609.4 |
| 356. | KCNE4 | NM_080671.3 |
| 357. | JAM3 | NM_032801.4 |
| 358. | SOBP | NM_018013.3 |
| 359. | LZTS1 | NM_021020.4 |
| 360. | STARD8 | NM_001142503.2 |
| 361. | RHOBTB3 | NM_014899.3 |
| 362. | ADAMTS4 | NM_005099.5 |
| 363. | COL4A1 | NM_001845.5 |
| 364. | COL4A2 | NM_001846.3 |
| 365. | SPRY4 | NM_030964.3 |
| 366. | MYADM | NM_001020818.2 |
| 367. | SLC39A14 | NM_001135153.1 |
| 368. | TBXA2R | NM_001060 |
| 369. | CD63 | NM_001780.5 |
| 370. | RUNX1 | NM_001754.4 |
| 371. | PRAF2 | NM_007213.2 |
| 372. | C1R | NM_001733.6 |
| 373. | GAB2 | NM_080491.2 |
| 374. | MMRN2 | NM_024756.2 |
| 375. | CCL2 | NM_002982.3 |
| 376. | FMOD | NM_002023.4 |
| 377. | CLEC10A | NM_001330070.1 |
| 378. | GIMAP8 | NM_175571.3 |
| 379. | CLEC14A | NM_175060.2 |
| 380. | ECSCR | NM_001077693.3 |
| 381. | ACVRL1 | NM_000020.2 |
| 382. | PECAM1 | NM_000442.4 |
| 383. | PCDH12 | NM_016580.3 |
| 384. | HLX | NM_021958.3 |
| 385. | C1QA | NM_015991.3 |
| 386. | CD14 | NM_000591.3 |
| 387. | TMEM176A | NM_018487.2 |
| 388. | TMEM176B | NM_014020.3 |
| 389. | STAB1 | NM_015136.2 |
| 390. | RNASE1 | NM_198235.2 |
| 391. | SERPING1 | NM_000062.2 |
| 392. | ACSL5 | NM_016234.3 |
| 393. | APOL3 | NM_014349.2 |
| 394. | PSMB10 | NM_002801.3 |
| 395. | UBA7 | NM_003335 |
| 396. | ARHGAP4 | NM_001164741.1 |
| 397. | MEI1 | NM_152513.3 |
| 398. | MTMR14 | NM_001077526.2 |
| 399. | FYB | NM_001465.4 |
| 400. | CCL19 | NM_006274.2 |
| 401. | LTB | NM_002341.1 |
| 402. | FGD2 | NM_173558 |
| 403. | ALOX5 | NM_000698.4 |
| 404. | ARHGDIB | NM_001175.6 |
| 405. | CD74 | NM_001025159 |
| 406. | HLA-DMA | NM_006120 |
| 407. | HLA-DRA | NM_019111.4 |
| 408. | HLA-DPA1 | NM_033554 |
| 409. | HLA-DPB1 | NM_002121.5 |
| 410. | HLA-DQB1 | NM_002123.4 |
| 411. | HLA-DQB2 | NM_001300790.1 |
| 412. | HLA-DRB5 | NM_002125 |
| 413. | APOB48R | NM_018690.3 |
| 414. | ABI3 | NM_016428.2 |
| 415. | LST1 | NM_007161.3 |
| 416. | MYO1F | NM_012335.3 |
| 417. | FGL2 | NM_006682.2 |
| 418. | GIMAP4 | NM_018326.2 |
| 419. | CIITA | NM_001286402 |
| 420. | HLA-DMB | NM_002118.4 |
| 421. | IRF8 | NM_001363907.1 |
| 422. | TNFAIP8L2 | NM_024575.4 |
| 423. | CCR5 | NM_000579.3 |
| 424. | CD2 | NM_001328609.1 |
| 425. | IL2RB | NM_000878.4 |
| 426. | SIRPG | NM_018556.3 |
| 427. | CCR7 | NM_001838.3 |
| 428. | NAPSB | NR_002798.1 |
| 429. | GIMAP5 | NM_018384.4 |
| 430. | WDFY4 | NM_020945.1 |
| 431. | INPP5D | NM_001017915.2 |
| 432. | CD6 | NM_006725.4 |
| 433. | ICAM3 | NM_002162.4 |
| 434. | GMFG | NM_004877.3 |
| 435. | RASSF2 | NM_014737.2 |
| 436. | IL10RA | NM_001558.3 |
| 437. | SELPLG | NM_001206609.1 |
| 438. | CD52 | NM_001803.2 |
| 439. | CD48 | NM_001778.3 |
| 440. | SASH3 | NM_018990.3 |
| 441. | PPP1R16B | NM_015568.3 |
| 442. | RASAL3 | NM_022904.2 |
| 443. | TBC1D10C | NM_198517.3 |
| 444. | TRAF3IP3 | NM_001320144.1 |
| 445. | IL21R | NM_181079.4 |
| 446. | RASSF4 | NM_032023.3 |
| 447. | C2 | NM_000063.5 |
| 448. | AIF1 | NM_032955.2 |
| 449. | CYBB | NM_000397.3 |
| 450. | TRPV2 | NM_016113.4 |
| 451. | LAT2 | NM_032464.2 |
| 452. | LY86 | NM_004271.3 |
| 453. | RNASE6 | NM_005615.4 |
| 454. | IL4I1 | NM_152899.1 |
| 455. | CXCR4 | NM_001008540.2 |
| 456. | WIPF1 | NM_003387.4 |
| 457. | TNFSF12 | NM_003809.2 |
| 458. | VASH1 | NM_014909.4 |
| 459. | DOCK8 | NM_203447.3 |
| 460. | SLAMF7 | NM_021181 |
| 461. | LSP1 | NM_002339.2 |
| 462. | PLEKHO2 | NM_025201.4 |
| 463. | TMEM140 | NM_018295.4 |
| 464. | VAMP5 | NM_006634.2 |
| 465. | FOXP1 | NM_032682.5 |
| 466. | KCTD12 | NM_138444.3 |
| 467. | RFTN1 | NM_015150 |
| 468. | CPNE5 | NM_020939.1 |
| 469. | ICAM2 | NM_001099786.1 |
| 470. | IL3RA | NM_001267713.1 |
| 471. | GRASP | NM_181711.3 |
| 472. | KLF2 | NM_016270.3 |
| 473. | RGS16 | NM_002928.3 |
| 474. | FAM110B | NM_147189.2 |
| 475. | FZD4 | NM_012193.3 |
| 476. | AOC3 | NM_003734.3 |
| 477. | COL14A1 | NM_021110.3 |
| 478. | CXCL12 | NM_199168.3 |
| 479. | JAM2 | NM_021219.3 |
| 480. | SOD3 | NM_003102.2 |
| 481. | FBLN5 | NM_006329.3 |
| 482. | MGP | NM_001190839.2 |
| 483. | CELF2 | NM_001025076.2 |
| 484. | ITM2A | NM_004867.4 |
| 485. | MEF2C | NM_001308002.1 |
| 486. | IFFO1 | NM_080730.4 |
| 487. | SYNE1 | NM_182961.3 |
| 488. | RCAN2 | NM_005822.3 |
| 489. | ZBTB47 | NM_145166.3 |
| 490. | ME3 | NM_006680.2 |
| 491. | IGFBP4 | NM_001552.2 |
| 492. | PLEKHO1 | NM_016274.5 |
| 493. | S100A4 | NM_002961.2 |
| 494. | PTP4A3 | NM_032611.2 |
| 495. | BATF3 | NM_018664.2 |
| 496. | CD97 | NM_078481.3 |
| 497. | C1orf38 | NM_004848.3 |
| 498. | NLRP3 | NM_004895.4 |
| 499. | CCRL2 | NM_003965.4 |
| 500. | SRGN | NM_002727.3 |
| 501. | TNFRSF1B | NM_001066.2 |
| 502. | PHC2 | NM_198040.2 |
| 503. | TSPAN4 | NM_001025237.1 |
| 504. | LHFPL2 | NM_005779.2 |
| 505. | DRAM1 | NM_018370.2 |
| 506. | SYTL3 | NM_001242384.1 |
| 507. | FAS | NM_000043.5 |
| 508. | FCER1A | NM_002001.3 |
| 509. | S100B | NM_006272.2 |
| 510. | C1orf123 | NM_017887.2 |
| 511. | CXXC5 | NM_001317199.1 |
| 512. | GSTK1 | NM_015917.2 |
| 513. | C10orf54 | NM_022153.1 |
| 514. | FAM40A | NM_033088 |
| 515. | C10orf26 | NM_001083913.1 |
| 516. | PINK1 | NM_032409.2 |
| 517. | JAK1 | NM_002227.3 |
| 518. | EPAS1 | NM_001430.4 |
| 519. | IL4R | NM_000418 |
| 520. | MBNL2 | NM_144778.3 |
| 521. | GRAMD3 | NM_026240.2 |
| 522. | ARHGAP1 | NM_004308.4 |
| 523. | STX12 | NM_177424.2 |
| 524. | ATP5O | NM_138597.2 |
| 525. | CMPK1 | NM_016308.2 |
| 526. | PDXK | NM_003681.4 |
| 527. | RPL22 | NM_000983.3 |
| 528. | ZFAND2B | NM_138802.2 |
| 529. | VAV3 | NM_006113 |
| 530. | LGALS9 | NM_009587.2 |
| 531. | LGALS9C | NM_001040078.2 |
| 532. | TFEB | NM_007162.2 |
| 533. | TMEM51 | NM_001136216 |
| 534. | DNMT3B | NM_006892.3 |
| 535. | HOMER3 | NM_001145722.1 |
| 536. | C9orf117 | NM_001012502.2 |
| 537. | C9orf21 | NM_153698.1 |
| 538. | CDK6 | NM_001259.7 |
| 539. | FKBP9 | NM_007270 |
| 540. | ACOT9 | NM_001037171.1 |
| 541. | CD276 | NM_001024736 |
| 542. | CSGALNACT2 | NM_018590.4 |
| 543. | ABL2 | NM_007314.3 |
| 544. | LGALS1 | NM_002305.3 |
| 545. | P4HA2 | NM_004199.2 |
| 546. | ACTN1 | NM_001130004 |
| 547. | INHBA | NM_002192 |
| 548. | SERPINE1 | NM_000602 |
| 549. | CAV1 | NM_001753 |
| 550. | TNFRSF12A | NM_016639.2 |
| 551. | FSTL3 | NM_005860 |
| 552. | MT1L | NR_001447.2 |
| 553. | MT2A | NM_005953.4 |
| 554. | LRRC8C | NM_032270.4 |
| 555. | NT5E | NM_002526.3 |
| 556. | APBB2 | NM_001166054.1 |
| 557. | VEGFC | NM_005429 |
| 558. | THBS1 | NM_003246.3 |
| 559. | GALNT10 | NM_198321.3 |
| 560. | GALNT2 | NM_004481.4 |
| 561. | TGFBI | NM_000358 |
| 562. | SNAI2 | NM_003068 |
| 563. | FMNL2 | NM_052905.3 |
| 564. | MSN | NM_002444.2 |
| 565. | SMTN | NM_134270.2 |
| 566. | CLIC4 | NM_013943.2 |
| 567. | MMP1 | NM_002421 |
| 568. | PDPN | NM_006474.4 |
| 569. | PTRF | NM_012232 |
| 570. | TGFB1 | NM_000660.6 |
| 571. | GNA12 | NM_007353.2 |
| 572. | TTYH3 | NM_025250.2 |
| 573. | PLAU | NM_002658.4 |
| 574. | PTK7 | NM_002821.4 |
| 575. | PTPRE | NM_006504.5 |
| 576. | REEP3 | NM_001001330.2 |
| 577. | SFXN3 | NM_030971 |
| 578. | SIRPA | NM_001040022.1 |
| 579. | ZBTB43 | NM_014007.3 |
| 580. | CARS | NM_139273.3 |
| 581. | TRIM5 | NM_033034.2 |
| 582. | CASP4 | NM_001225 |
| 583. | TMEM86A | NM_153347.2 |
| 584. | GPBAR1 | NM_001077191.1 |
| 585. | MOBKL2B | NM_024761 |
| 586. | HNRNPF | NM_001098208.1 |
| 587. | PSMA5 | NM_002790.3 |
| 588. | ACTR1A | NM_005736 |
| 589. | ADK | NM_001123.3 |
| 590. | SLC31A2 | NM_001860 |
| 591. | TAOK3 | NM_001346487.1 |
| 592. | VDR | NM_001364085.1 |
| 593. | STK19 | NM_004197.1 |
| 594. | CDS1 | NM_001263.3 |
| 595. | ARHGAP32 | NM_001142685.1 |
| 596. | FAM135A | NM_001105531.2 |
| 597. | BTRC | NM_033637.3 |
| 598. | TCHP | NM_032300.4 |
| 599. | AQP3 | NM_004925.4 |
| 600. | DHRS1 | NM_001136050 |
| 601. | CLTB | NM_001834.3 |
| 602. | FAM46B | XM_427638.6 |
| 603. | KCNK6 | NM_004823.2 |
| 604. | CLIC3 | NM_004669.2 |
| 605. | AIM1 | NM_119045.5 |
| 606. | RAB27B | NM_004163.4 |
| 607. | ATP10D | NM_020453.3 |
| 608. | IL18 | NM_001562.3 |
| 609. | NDFIP2 | NM_019080.2 |
| 610. | GSDMC | NM_031415.2 |
| 611. | GPR115 | AY140957.1 |
| 612. | SAMD9 | NM_017654.3 |
| 613. | WNT4 | NM_030761.4 |
| 614. | PALMD | NM_017734.4 |
| 615. | FBXO3 | NM_012175.3 |
| 616. | CTR9 | NM_014633.4 |
| 617. | RNF141 | NM_016422.3 |
| 618. | ABCA12 | NM_173076.2 |
| 619. | D4S234E | NM_014392.4 |
| 620. | DSP | NM_004415.3 |
| 621. | DSC2 | NM_024422.4 |
| 622. | IL1F5 | NM_001146087.1 |
| 623. | HSPC159 | NM_014181.2 |
| 624. | KRT75 | NM_004693.2 |
| 625. | IL22RA1 | NM_021258.3 |
| 626. | SERPINB5 | NM_002639.4 |
| 627. | CSNK1A1 | NM_001025105 |
| 628. | CSNK1A1L | NM_145203.5 |
| 629. | GM2A | NM_000405.4 |
| 630. | SERPINB8 | NM_002640.3 |
| 631. | CLPX | NM_006660.4 |
| 632. | TMEM154 | NM_152680.2 |
| 633. | TMOD3 | NM_014547.4 |
| 634. | BCL2L2 | NM_004050.4 |
| 635. | CPEB2 | NM_182646.2 |
| 636. | PLD2 | NM_002663 |
| 637. | RREB1 | NM_001003699.3 |
| 638. | EEA1 | NM_003566.3 |
| 639. | RCOR1 | NM_015156.3 |
| 640. | FAM83B | NM_001010872.2 |
| 641. | SLC10A6 | NM_197965.2 |
| 642. | SOX15 | NM_006942.1 |
| 643. | CDSN | NM_001264 |
| 644. | GSDMA | NM_178171 |
| 645. | DSG1 | NM_001942 |
| 646. | RFX1 | NM_002918.4 |
| 647. | NXPH4 | NM_007224.3 |
| 648. | RIMS3 | NM_014747.3 |
| 649. | SERPINB7 | NM_003784.3 |
| 650. | ATP6V0D1 | NM_004691.4 |
| 651. | MAP4 | NM_002375.4 |
| 652. | KCTD5 | NM_018992.3 |
| 653. | PIP5KL1 | NM_001135219.1 |
| 654. | LUZP1 | NM_033631.3 |
| 655. | SPOPL | NM_001001664.2 |
| 656. | USP53 | NM_019050.2 |
| 657. | IMPDH1 | NM_000883.3 |
| 658. | ENAH | NM_001008493.2 |
| 659. | KIF13A | NM_022113.5 |
| 660. | GALNT6 | NM_007210 |
| 661. | MYO5A | NM_000259.3 |
| 662. | FAM89A | NM_198552.2 |
| 663. | SSFA2 | NM_001130445.2 |
| 664. | CAB39 | NM_016289 |
| 665. | BMP2K | NM_198892.1 |
| 666. | MALT1 | NM_006785.4 |
| 667. | MAP7D1 | NM_018067 |
| 668. | RRAGC | NM_022157.3 |
| 669. | VAMP3 | NM_004781 |
| 670. | HERC4 | NM_022079.2 |
| 671. | PGBD3 | NM_170753.3 |
| 672. | CDC42 | NM_001791.3 |
| 673. | ATP6V1D | NM_015994 |
| 674. | DAAM1 | NM_014992.2 |
| 675. | EIF2S1 | NM_004094.4 |
| 676. | MRPS10 | NM_018141.3 |
| 677. | CCRN4L | NM_012118.3 |
| 678. | FBLIM1 | NM_017556 |
| 679. | FSCN1 | NM_003088.3 |
| 680. | LPAR3 | NM_012152.2 |
| 681. | PTGFRN | NM_020440.3 |
| 682. | PLCB3 | NM_000932.2 |
| 683. | RPS6KA4 | NM_003942.2 |
| 684. | SERINC2 | NM_178865.4 |
| 685. | SLC9A1 | NM_003047.4 |
| 686. | TNKS1BP1 | NM_033396.2 |
| 687. | CEBPB | NM_005194.3 |
| 688. | CTSL1 | NM_001912.4 |
| 689. | C16orf57 | NM_024598 |
| 690. | LACTB | NM_032857.4 |
| 691. | SLC35D1 | NM_015139.2 |
| 692. | MICALCL | NM_032867.3 |
| 693. | MYO1B | NM-001130158.2 |
| 694. | NEDD4 | NM_006154.3 |
| 695. | THSD1 | NM_018676.3 |
| 696. | PANX1 | NM_015368.3 |
| 697. | RRAS2 | NM_012250.5 |
| 698. | RGS20 | NM_170587 |
| 699. | SH2D5 | NM_001103161 |
| 700. | SIN3B | NM_015260.3 |
| 701. | OTUD1 | NM_001145373.2 |
| 702. | TMCC3 | NM_020698.3 |
| 703. | DUSP14 | NM_007026.3 |
| 704. | ATP11A | NM_015205.2 |
| 705. | PPARD | NM_006238 |
| 706. | PPIF | NM_005729.3 |
| 707. | UPP1 | NM_003364.3 |
| 708. | KIAA1919 | BC036115.1 |
| 709. | PAQR5 | NM_001104554 |
| 710. | WDFY2 | NM_052950.3 |
| 711. | YWHAZ | NM_003406.3 |
| 712. | HRAS | NM_005343.3 |
| 713. | KIAA1609 | AB046829.2 |
| 714. | RAB22A | NM_020673.2 |
| 715. | PKP3 | NM_007183 |
| 716. | RAB38 | NM_022337.2 |
| 717. | C1orf113 | NM_001162530.1 |
| 718. | NDST1 | NM_001543.4 |
| 719. | ZCCHC6 | NM_024617.3 |
| 720. | PI4K2A | NM_018425.3 |
| 721. | VTI1A | NM_001318203.1 |
| 722. | RBM18 | NM_033117.3 |
| 723. | ROD1 | AB023967.1 |
| 724. | YWHAQ | NM_006826.3 |
| 725. | VANGL1 | NM_138959.2 |
| 726. | F2RL1 | NM_005242 |
| 727. | TMEM87B | NM_032824.2 |
| 728. | RNF152 | NM_173557.2 |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 4
14 gene subtype classifier for gene
expression based subtyping of HNSCC.

| Number | Gene Symbol | GenBank Accession Number* |
|---|---|---|
| 1 | AKR1C1 | NM_001353 |
| 2 | NFE2L2 | NM_006164 |
| 3 | SOX2 | NM_003106 |
| 4 | KEAP1 | NM_012289 |
| 5 | RPA2 | NM_001286076 |
| 6 | E2F2 | NM_004091 |
| 7 | FGFR3 | NM_000142 |
| 8 | PDGFRA | NM_006206.5 |
| 9 | PDGFRB | NM_002609.3 |
| 10 | TWIST1 | NM_000474.3 |
| 11 | EGFR | NM_001346897 |
| 12 | PIK3CA | NM_006218.3 |
| 13 | TP63 | NM_003722.4 |
| 14 | TGFA | NM_003236.3 |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

### Numbered Embodiments of the Disclosure

Other subject matter contemplated by the present disclosure is set out in the following numbered embodiments:

1. A method of determining a suitable treatment for a head and neck squamous cell carcinoma (HNSCC) patient, the method comprising: (a) detecting an expression level of at least one subtype classifier selected from Table 3 or Table 4 in a head and neck tissue sample obtained from the patient; and (b) selecting a treatment for the HNSCC patient according to the expression level of the at least one subtype classifier selected from Table 3 or Table 4, wherein the detection of the expression level of the subtype classifier specifically identifies a basal (BA), mesenchymal (MS), atypical (AT) or classical (CL) HNSCC subtype, and wherein the patient is HPV negative.

2. The method of embodiment 1, wherein the expression level of the classifier biomarker is detected at the nucleic acid level.

3. The method of embodiment 2, wherein the nucleic acid level is RNA or cDNA.

4. The method of embodiment 2 or 3, wherein the detecting the expression level comprises performing quantitative real time reverse transcriptase polymerase chain reaction (qRT-PCR), gRT-PCR, RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

5. The method of embodiment 3 or 4, wherein the expression level is detected by performing RNAseq.

6. The method of any of the above embodiments, wherein the expression level is determined by RNAseq by Expected Maximization (RSEM).

7. The method of any of embodiments 2-6, wherein the detecting the expression level comprises using at least one pair of oligonucleotide primers specific for at least one subtype classifier selected from Table 3 or Table 4.

8. The method of any of the above embodiments, wherein the sample is a formalin-fixed, paraffin-embedded (FFPE) head and neck tissue sample, fresh or a frozen tissue sample, an exosome, wash fluids, cell pellets, or a bodily fluid obtained from the patient.

9. The method of embodiment 8, wherein the bodily fluid is blood or fractions thereof, urine, saliva, or sputum.

10. The method of any one of the above embodiments, wherein the at least one subtype classifier comprises a plurality of subtype classifiers.

11. The method of any of embodiments 1-10, wherein the at least one subtype classifier comprises all the subtype classifiers of Table 3 or Table 4.

12. The method of embodiment 1, wherein the HNSCC is oral cavity squamous cell carcinoma (OCSCC).

13. The method of embodiment 1, wherein the HNSCC is laryngeal squamous cell carcinoma (LSCC).

14. The method of embodiment 12, wherein the OCSCC is the MS subtype.

15. The method of embodiment 12, wherein the OCSCC is the BA subtype.

16. The method of embodiment 13, wherein the LSCC is the CL subtype.

17. The method of embodiment 13, wherein the LSCC is the AT subtype.

18. The method of embodiment 1, wherein the treatment comprises radiotherapy or surgery.

19. The method of embodiment 1, further comprising identifying resistance to radiotherapy.

20. The method of embodiment 19, wherein the identifying comprises comparing the expression levels of the at least one subtype classifier selected from Table 3 or Table 4 to expression levels of the at least one subtype classifier selected from Table 3 or Table 4 in radiotherapy responder controls, radiotherapy non-responder controls or a combination thereof.

21. The method of embodiment 19, wherein the identifying comprises measuring expression level of one or more genes in the KEAP1/NRF2 pathway.

22. The method of embodiment 19, wherein the identifying comprises detecting a mutation in one or more genes in the KEAP1/NRF2 pathway.

23. The method of embodiment 14, wherein the MS subtype is predictive of pathological nodal metastasis.

24. The method of any of the above embodiments, wherein the subtype is predictive of overall survival of the patient.

25. The method of embodiment 24, wherein the CL subtype in LSCC is predictive of a poor overall survival.

26. The method of any of the above embodiments, wherein the at least one subtype classifier is selected from Table 3.

27. The method of embodiment 26, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

28. The method of any of embodiments 1-25, wherein the at least one subtype classifier is selected from Table 4.

29. A method of determining whether a HNSCC patient is likely to respond to radiotherapy, the method comprising: (a) detecting an expression level of at least one subtype classifier selected from Table 3 or Table 4 in a head and neck tissue sample obtained from the patient, wherein the patient is HPV negative, and wherein the detection of the expression level of the subtype classifier specifically identifies a BA, MS, AT or CL HNSCC subtype; (b) determining expression of one or more genes associated with radiotherapy resistance; and (c) identifying the HNSCC subtype correlated with radiotherapy resistance.

30. The method of embodiment 29, wherein the expression level of the subtype classifier is detected at the nucleic acid level.

31. The method of embodiment 30, wherein the nucleic acid level is RNA or cDNA.

32. The method of embodiment 30 or 31, wherein the detecting the expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

33. The method of embodiment 31 or 32, wherein the expression level is detected by performing RNAseq.

34. The method of any of embodiments 29-33, wherein the expression level is determined by RSEM.

35. The method of any of embodiments 30-34, wherein the detecting the expression level comprises using at least one pair of oligonucleotide primers specific for the at least one subtype classifier selected from Table 3 or Table 4.

36. The method of embodiments 29-35, wherein the sample is a FFPE head and neck tissue sample, fresh or a frozen tissue sample, an exosome, wash fluids, cell pellets, or a bodily fluid obtained from the patient.

37. The method of embodiment 36, wherein the bodily fluid is blood or fractions thereof, urine, saliva, or sputum.

38. The method of embodiments 29-37, wherein the at least one subtype classifier comprises a plurality of subtype classifiers.

39. The method of any of embodiments 29-38, wherein the at least one subtype classifier comprises all the subtype classifiers of Table 3 or Table 4.

40. The method of embodiment 29, wherein the HNSCC is OCSCC.

41. The method of embodiment 29, wherein the HNSCC is LSCC.

42. The method of embodiment 40, wherein the OCSCC is the MS subtype.

43. The method of embodiment 40, wherein the OCSCC is the BA subtype.

44. The method of embodiment 41, wherein the LSCC is the CL subtype.

45. The method of embodiment 41, wherein the LSCC is the AT subtype.

46. The method of embodiment 29, wherein the HNSCC is the CL subtype.

47. The method of embodiment 29, further comprising comparing the expression levels of the at least one subtype classifier selected from Table 3 or Table 4 between expression levels of the at least one subtype classifier selected from Table 3 or Table 4 in radiotherapy responder controls and/or expression levels of the at least one subtype classifier selected from Table 3 or Table 4 in radiotherapy non-responder controls.

48. The method of embodiment 29, wherein the identifying comprises measuring expression level of one or more genes in the KEAP1/NRF2 pathway.

49. The method of embodiment 29, wherein the identifying comprises detecting a mutation in one or more genes in the KEAP1/NRF2 pathway.

50. The method of any of embodiments 29-49, wherein the at least one subtype classifier is selected from Table 3.

51. The method of embodiment 50, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

52. The method of embodiments 29-49, wherein the at least one subtype classifier is selected from Table 4.

53. A method of predicting occult nodal metastasis in a OCSCC patient, the method comprising: (a) detecting an expression level of at least one gene selected from Table 3 or Table 4 in a head and neck tissue sample obtained from a patient, wherein the patient is HPV negative, wherein the detection of the expression level of the subtype classifier specifically identifies a BA, MS, AT or CL HNSCC subtype, and wherein identification of the MS subtype is indicative of occult nodal metastasis in the patient.

54. The method of embodiment 53, wherein the expression level of the classifier biomarker is detected at the nucleic acid level.

55. The method of embodiment 54, wherein the nucleic acid level is RNA or cDNA.

56. The method embodiment 54 or 55, wherein the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

57. The method of embodiment 55 or 56, wherein the expression level is detected by performing RNAseq.

58. The method of any of embodiments 53-57, wherein the expression level is determined by RSEM.

59. The method of any of embodiments 54-58, wherein the detection of the expression level comprises using at least one pair of oligonucleotide primers specific for at least one subtype classifier selected from Table 3 or Table 4.

60. The method of any of embodiments 54-59, wherein the sample is a FFPE head and neck tissue sample, fresh or a frozen tissue sample, an exosome, wash fluids, cell pellets, or a bodily fluid obtained from the patient.

61. The method of embodiment 60, wherein the bodily fluid is blood or fractions thereof, urine, saliva, or sputum.

62. The method of any of embodiments 53-61, wherein the at least one subtype classifier comprises a plurality of subtype classifiers.

63. The method of any of embodiments 53-62, wherein the at least one subtype classifier comprises all the subtype classifiers of Table 3 or Table 4.

64. The method of embodiment 53, wherein the patient is suitable for neck dissection treatment.

65. The method of any of embodiments 53-64, wherein the at least one subtype classifier is selected from Table 3.

66. The method of embodiment 65, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

67. The method of embodiments 53-64, wherein the at least one subtype classifier is selected from Table 4.

68. A method of predicting overall survival in a LSCC patient, the method comprising detecting an expression level of at least one gene selected from Table 3 or Table 4 in a head and neck tissue sample obtained from a patient, wherein the patient is HPV negative, wherein the detection of the expression level of the subtype classifier specifically identifies a BA, MS, AT or CL LSCC subtype, and wherein identification of the LSCC subtype is predictive of the overall survival in the patient.

69. The method of embodiment 68, wherein the expression level of the classifier biomarker is detected at the nucleic acid level.

70. The method of embodiment 69, wherein the nucleic acid level is RNA or cDNA.

71. The method embodiment 69 or 70, wherein the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

72. The method of embodiment 70 or 71, wherein the expression level is detected by performing RNAseq.

73. The method of any of embodiments 68-72, wherein the expression level is determined by RSEM.

74. The method of any of embodiments 69-73, wherein the detection of the expression level comprises using at least one pair of oligonucleotide primers specific for at least one subtype classifier selected from Table 3 or Table 4.

75. The method of any of embodiments 68-74, wherein the sample is a FFPE head and neck tissue sample, fresh or a frozen tissue sample, an exosome, wash fluids, cell pellets, or a bodily fluid obtained from the patient.

76. The method of embodiment 75, wherein the bodily fluid is blood or fractions thereof, urine, saliva, or sputum.

77. The method of any of embodiments 68-76, wherein the at least one subtype classifier comprises a plurality of subtype classifiers.

78. The method of any of embodiments 68-77, wherein the at least one subtype classifier comprises all the subtype classifiers of Table 3 or Table 4.

79. The method of any of embodiments 68-78, further comprising measuring the expression level of one or more genes in the KEAP1/NRF2 pathway.

80. The method of any of embodiments 68-78, further comprising detecting a mutation in one or more genes in the KEAP1/NRF2 pathway.

81. The method of any of embodiments 68-80, wherein the LSCC subtype is the CL subtype, wherein the CL subtype is predictive of poor overall survival.

82. The method of embodiment 81, wherein the patient is suitable for neck dissection treatment.

83. The method of any of embodiments 68-82, wherein the at least one subtype classifier is selected from Table 3.

84. The method of embodiment 83, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

85. The method of embodiments 68-82, wherein the at least one subtype classifier is selected from Table 4.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of determining a suitable treatment for a head and neck squamous cell carcinoma (HNSCC) patient, the method comprising: (a) detecting an expression level of at least one subtype classifier selected from Table 3 or Table 4 in a head and neck tissue sample obtained from the patient; and (b) selecting a treatment for the HNSCC patient according to the expression level of the at least one subtype classifier selected from Table 3 or Table 4; wherein the detection of the expression level of the subtype classifier specifically identifies a basal (BA), mesenchymal (MS), atypical (AT) or classical (CL) HNSCC subtype, and wherein the patient is HPV negative.

2. The method of claim 1, wherein the expression level of the classifier biomarker is detected at the nucleic acid level.

3. The method of claim 2, wherein the nucleic acid level is RNA or cDNA.

4. The method of claim 2, wherein the detecting the expression level comprises performing quantitative real time reverse transcriptase polymerase chain reaction (qRT-PCR), gRT-PCR, RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

5. The method of claim 4, wherein the expression level is detected by performing RNAseq.

6. The method of claim 5, wherein the expression level is determined by RNAseq by Expected Maximization (RSEM).

7. The method of claim 2, wherein the detecting the expression level comprises using at least one pair of oligonucleotide primers specific for at least one subtype classifier selected from Table 3 or 4.

8. The method of claim 1, wherein the sample is a formalin-fixed, paraffin-embedded (FFPE) head and neck tissue sample, fresh or a frozen tissue sample, an exosome, wash fluids, cell pellets, or a bodily fluid obtained from the patient.

9. The method of claim 8, wherein the bodily fluid is blood or fractions thereof, urine, saliva, or sputum.

10. The method of claim 1, wherein the at least one subtype classifier comprises a plurality of subtype classifiers.

11. The method of claim 1, wherein the at least one subtype classifier comprises all the subtype classifiers of Table 3 or Table 4.

12. The method of claim 1, wherein the HNSCC is oral cavity squamous cell carcinoma (OCSCC).

13. The method of claim 1, wherein the HNSCC is laryngeal squamous cell carcinoma (LSCC).

14. The method of claim 12, wherein the OCSCC is the MS subtype.

15. The method of claim 12, wherein the OCSCC is the BA subtype.

16. The method of claim 13, wherein the LSCC is the CL subtype.

17. The method of claim 13, wherein the LSCC is the AT subtype.

18. The method of claim 1, wherein the treatment comprises radiotherapy or surgery.

19. The method of claim 1, further comprising identifying resistance to radiotherapy.

20. The method of claim 19, wherein the identifying comprises comparing the expression levels of the at least one subtype classifier selected from Table 3 or Table 4 to expression levels of the at least one subtype classifier selected from Table 3 or Table 4 in radiotherapy responder controls, radiotherapy non-responder controls or a combination thereof.

21. The method of claim 19, wherein the identifying comprises measuring expression level of one or more genes in the KEAP1/NRF2 pathway.

22. The method of claim 19, wherein the identifying comprises detecting a mutation in one or more genes in the KEAP1/NRF2 pathway.

23. The method of claim 14, wherein the MS subtype is predictive of pathological nodal metastasis.

24. The method of claim 1, wherein the subtype is predictive of overall survival of the patient.

25. The method of claim 24, wherein the CL subtype in LSCC is predictive of a poor overall survival.

26. The method of claim 1, wherein the at least one subtype classifier is selected from Table 3.

27. The method of claim 26, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

28. The method of claim 1, wherein the at least one subtype classifier is selected from Table 4.

29. A method of determining whether a HNSCC patient is likely to respond to radiotherapy, the method comprising: (a) detecting an expression level of at least one subtype classifier selected from Table 3 or Table 4 in a head and neck tissue sample obtained from the patient, wherein the patient is HPV negative, and wherein the detection of the expression level of the subtype classifier specifically identifies a BA, MS, AT or CL HNSCC subtype; (b) determining expression of one or more genes associated with radiotherapy resistance; and (c) identifying the HNSCC subtype correlated with radiotherapy resistance.

30. The method of claim 29, wherein the expression level of the subtype classifier is detected at the nucleic acid level.

31. The method of claim 30, wherein the nucleic acid level is RNA or cDNA.

32. The method of claim 30, wherein the detecting the expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

33. The method of claim 32, wherein the expression level is detected by performing RNAseq.

34. The method of claim 33, wherein the expression level is determined by RSEM.

35. The method of claim 30, wherein the detecting the expression level comprises using at least one pair of oligonucleotide primers specific for the at least one subtype classifier selected from Table 3 or Table 4.

36. The method of claim 29, wherein the sample is a FFPE head and neck tissue sample, fresh or a frozen tissue sample, an exosome, wash fluids, cell pellets, or a bodily fluid obtained from the patient.

37. The method of claim 36, wherein the bodily fluid is blood or fractions thereof, urine, saliva, or sputum.

38. The method of claim 29, wherein the at least one subtype classifier comprises a plurality of subtype classifiers.

39. The method of claim 29, wherein the at least one subtype classifier comprises all the subtype classifiers of Table 3 or Table 4.

40. The method of claim 29, wherein the HNSCC is OCSCC.

41. The method of claim 29, wherein the HNSCC is LSCC.

42. The method of claim 40, wherein the OCSCC is the MS subtype.

43. The method of claim 40, wherein the OCSCC is the BA subtype.

44. The method of claim 41, wherein the LSCC is the CL subtype.

45. The method of claim 41, wherein the LSCC is the AT subtype.

46. The method of claim 29, wherein the HNSCC is the CL subtype.

47. The method of claim 29, further comprising comparing the expression levels of the at least one subtype classifier selected from Table 3 or Table 4 between expression levels of the at least one subtype classifier selected from Table 3 or Table 4 in radiotherapy responder controls and/or expression levels of the at least one subtype classifier selected from Table 3 or Table 4 in radiotherapy non-responder controls.

48. The method of claim 29, wherein the identifying comprises measuring expression level of one or more genes in the KEAP1/NRF2 pathway.

49. The method of claim 29, wherein the identifying comprises detecting a mutation in one or more genes in the KEAP1/NRF2 pathway.

50. The method of claim 29, wherein the at least one subtype classifier is selected from Table 3.

51. The method of claim 50, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

52. The method of claim 29, wherein the at least one subtype classifier is selected from Table 4.

53. A method of predicting occult nodal metastasis in a OCSCC patient, the method comprising: (a) detecting an expression level of at least one gene selected from Table 3 or Table 4 in a head and neck tissue sample obtained from a patient, wherein the patient is HPV negative, wherein the detection of the expression level of the subtype classifier specifically identifies a BA, MS, AT or CL HNSCC subtype, and wherein identification of the MS subtype is indicative of occult nodal metastasis in the patient.

54. The method of claim 53, wherein the expression level of the classifier biomarker is detected at the nucleic acid level.

55. The method of claim 54, wherein the nucleic acid level is RNA or cDNA.

56. The method claim 54, wherein the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

57. The method of claim 56, wherein the expression level is detected by performing RNAseq.

58. The method of claim 53, wherein the expression level is determined by RSEM.

59. The method of claim 54, wherein the detection of the expression level comprises using at least one pair of oligonucleotide primers specific for at least one subtype classifier selected from Table 3 or Table 4.

60. The method of claim 54, wherein the sample is a FFPE head and neck tissue sample, fresh or a frozen tissue sample, an exosome, wash fluids, cell pellets, or a bodily fluid obtained from the patient.

61. The method of claim 60, wherein the bodily fluid is blood or fractions thereof, urine, saliva, or sputum.

62. The method of claim 53, wherein the at least one subtype classifier comprises a plurality of subtype classifiers.

63. The method of claim 53, wherein the at least one subtype classifier comprises all the subtype classifiers of Table 3 or Table 4.

64. The method of claim 53, wherein the patient is suitable for neck dissection treatment.

65. The method of claim 53, wherein the at least one subtype classifier is selected from Table 3

66. The method of claim 65, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

67. The method of claim 53, wherein the at least one subtype classifier is selected from Table 4.

68. A method of predicting overall survival in a LSCC patient, the method comprising detecting an expression level of at least one gene selected from Table 3 or Table 4 in a head and neck tissue sample obtained from a patient, wherein the patient is HPV negative, wherein the detection of the expression level of the subtype classifier specifically identifies a BA, MS, AT or CL LSCC subtype, and wherein identification of the LSCC subtype is predictive of the overall survival in the patient.

69. The method of claim 68, wherein the expression level of the classifier biomarker is detected at the nucleic acid level.

70. The method of claim 69, wherein the nucleic acid level is RNA or cDNA.

71. The method claim 69, wherein the detecting an expression level comprises performing qRT-PCR, gRT-PCR, RNAseq, microarrays, gene chips, nCounter Gene Expression Assay, SAGE, RAGE, nuclease protection assays, Northern blotting, or any other equivalent gene expression detection techniques.

72. The method of claim 71, wherein the expression level is detected by performing RNAseq.

73. The method of claim 72, wherein the expression level is determined by RSEM.

74. The method of claim 69, wherein the detection of the expression level comprises using at least one pair of oligonucleotide primers specific for at least one subtype classifier selected from Table 3 or Table 4.

75. The method of claim 68, wherein the sample is a FFPE head and neck tissue sample, fresh or a frozen tissue sample, an exosome, wash fluids, cell pellets, or a bodily fluid obtained from the patient.

76. The method of claim 75, wherein the bodily fluid is blood or fractions thereof, urine, saliva, or sputum.

77. The method of claim 68, wherein the at least one subtype classifier comprises a plurality of subtype classifiers.

78. The method of claim 68, wherein the at least one subtype classifier comprises all the subtype classifiers of Table 3 or Table 4.

79. The method of claim 68, further comprising measuring the expression level of one or more genes in the KEAP1/NRF2 pathway.

80. The method of claim 68, further comprising detecting a mutation in one or more genes in the KEAP1/NRF2 pathway.

81. The method of claim 68, wherein the LSCC subtype is the CL subtype, wherein the CL subtype is predictive of poor overall survival.

82. The method of claim 81, wherein the patient is suitable for neck dissection treatment.

83. The method of claim 68, wherein the at least one subtype classifier is selected from Table 3.

84. The method of claim 83, wherein the plurality of subtype classifiers comprises at least 2 subtype classifiers, at least 10 subtype classifiers, at least 50 subtype classifiers, at least 100 subtype classifiers, at least 200 subtype classifiers, at least 300 subtype classifiers, at least 400 subtype classifiers, at least 500 subtype classifiers, at least 600 subtype classifiers, at least 700 subtype classifiers, or all 728 subtype classifiers of Table 3.

85. The method of claim 68, wherein the at least one subtype classifier is selected from Table 4.
